(19) United States
(12) Patent Application Publication
(10) Pub. No.: US 2026/0125713 A1
(43) Pub. Date: May 7, 2026

(54) PRODUCTION OF MOLECULES BY PERIPLASMIC ENZYMATIC CATALYSIS

(71) Applicant: BIOC3, Toulouse Cedex 04 (FR)

(72) Inventors: Cléa LACHAUX, Toulouse Cedex 04 (FR); Eric GRONDIN, Toulouse Cedex 04 (FR); Yvan CAM, Toulouse Cedex 04 (FR)

(73) Assignee: BIOC3, Toulouse Cedex 04 (FR)

(21) Appl. No.: 18/862,650

(22) PCT Filed: May 4, 2023
(86) PCT No.: PCT/EP2023/061870

§ 371 Date: Nov. 4, 2024

(30) Foreign Application Priority Data
May 6, 2022 (FR) FR2204333

(51) Int. Cl. C12P 1/04; C12N 1/20; C12P 39/00; C12R 1/01

(52) U.S. Cl. CPC C12P 1/04; C12N 1/20; C12P 39/00; C12R 2001/01

(57) ABSTRACT

A process for producing a compound of interest from an organic substrate, including the steps of: a) preparing a reaction mixture including at least two bacteria genetically modified to express, in their periplasmic space, at least one enzyme, each of said bacteria expressing an enzyme different from the other bacteria, an enzyme being capable of catalyzing a first reaction using said organic substrate so as to provide a first product, each of the enzymes being capable of catalyzing a reaction using a product or a coproduct obtained by a reaction, so as to provide respectively a product, b) leaving the reaction mixture to react, and c) separating the biomass from the supernatant and extracting therefrom said compound of interest consisting of one of the products.

The present invention belongs to the field of production of molecules of interest by genetically modified microorganisms, and more particularly concerns the production of compounds by enzymatic catalysis conducted in the bacterial periplasmic space.

It relates to a process for the production of a compound of interest involving a mixture of bacteria (consortium), modified to express one or more enzymes in their periplasmic space, which together catalyze a reaction cascade for the production of a compound of interest. The invention also relates to a reaction mixture for the production of such a compound and a kit for preparing said medium.

The production of organic compounds by the petrochemical industry raises crucial future problems in terms of cost and environmental impact issues, especially due to the large amounts of carbon released into the atmosphere and the use of non-renewable resources. The production of such compounds by biological pathways appears as a non-polluting and sustainable alternative. It is still necessary to be able to carry out the appropriate reaction schemes and to obtain sufficient quantities in relation to the applications envisaged, according to economically acceptable protocols. Indeed, while some compounds can be obtained by a simple reaction, others require intermediate steps that complicate and increase the cost of the process. In addition, many reactions use cofactors which must be introduced into the reaction medium in a quantity proportional to the substrate to be transformed, which increases the costs accordingly.

Currently, various technologies using biological processes are used for the production of molecules. The most classic is the extraction of products of interest, for example flavors or therapeutic molecules, from organisms such as plants, by infusion maceration, extraction by solvent, or other means. However, plant extraction is limited in its applications, because it generally has fairly low yields compared to the mass processed. It uses complex processes to isolate and purify the molecule of interest. Finally, the seasonal or geographical availability of the source organism greatly constrains extraction strategies.

Another technique, also well known, is based on the fermentative activity of microorganisms that are may or may not be genetically modified, whose cellular metabolism produces molecules of interest, secreted in a culture medium. The production of alcohol by yeasts is one of the oldest examples. Fermentation has certain advantages. It can be carried out all year round at a moderate cost, since the reactions are carried out by microorganisms. Theoretical yields are generally interesting, as the contribution of synthetic biology and systems biology has greatly increased the efficiency of this type of system. Nevertheless, fermentation has notable disadvantages arising from the living nature of the biocatalyst. Thus, in practice, theoretical yields are difficult to obtain, since the cell always tends to redirect the flows engaged in the production of the molecule of interest toward its biomass. Moreover, the molecule of interest is secreted into a fermentation medium necessarily containing a large number of other molecules, which complicates its separation and consumes a large amount of water that must then be treated. Furthermore, not all molecules can be produced by fermentation, either because of their toxicity to the cell or because little to none of the molecule is excreted. Added to these difficulties is the development time of an industrial fermentative organism, of about ten years on average. Thus, several limitations of production by fermentation can be identified: i) the coupling of growth and production limits the maximum yield of production, ii) the use of synthetic biology to engineer a microorganism and redirect the carbon flow to the product is done at the expense of growth, which impacts the robustness of the process, iii) the operating conditions are limited to conditions compatible with microbial growth (pH, temperature, composition of the medium, oxygenation, etc.), iv) the substrates and products must not be cytotoxic; the maximum concentration of the substrate and the product are therefore fixed according to their toxicity, v) the substrate and product must be able to cross the cytoplasmic membrane, and vi) the product is secreted into a culture medium loaded with organic acids and other by-products, complicating the purification of the product and increasing the overall cost of the process.

More recently, whole cell bioproduction techniques have made it possible to overcome several of these disadvantages. Growth (enzyme production phase) and production are decoupled; the substrate is only dedicated to product synthesis. The operating conditions can be optimized to maximize production; compatibility with growth is not required. In addition, the purification of the product is simplified because the product is secreted in a simple medium free of growth coproducts. Finally, if the transfer of the substrate/product through the cytoplasmic membrane is limiting, one way of circumventing this limitation consists of rendering the cell wall and the membranes permeable by a chemical treatment (for example by adding detergents or solvents) or a physical treatment (for example by using a thermal shock). Nevertheless, these procedures can interfere with downstream purification processes, in addition to causing cellular damage. In addition, the main limitation of such a process is the need for metabolic engineering to i) optimize the expression level of the gene(s) coding for the enzyme(s) of the production pathway and ii) minimize intracellular side reactions. However optimizing the amount of each enzyme in an enzyme cascade is crucial for the overall performance of the process. Indeed, each enzyme in a metabolic pathway is responsible for a specific biochemical reaction necessary for the synthesis of a final product. If an insufficient quantity of an enzyme is present in the cascade, upstream substrates may accumulate, resulting in a reduction in the reaction rate and, consequently, in the production of the final product. In return, an excessive quantity of an enzyme can also disrupt the balance of the enzyme cascade and thus reduce the efficiency of the entire process. Consequently, the value of optimizing the quantity of each enzyme is understood in order to maximize the overall reaction efficiency and the production of the final product.

While it is possible to easily adjust the concentration of purified enzymes in the reaction mixture, in return, when these enzymes are expressed in vivo, regulation of their gene expression is necessary to achieve the optimal level of each enzyme. Currently, metabolic engineering and synthetic biology are used to improve yields and titers of products. Despite the constant development of new metabolic engineering tools, it is difficult to predict the level of enzyme production in the cell. The variation in the level of expression of a gene depends on several parameters including the number of copies of the gene—determined by the origin of replication when it is carried by an expression plasmid, the level of transcription determined by the choice of promoter and the level of translation by the ribosome binding site (RBS). However, the correlation between the efficiency of the expression system and the number of copies, transcription or translation force, is not linear. High expression can represent a significant cellular metabolic load, to the detriment of production and growth. Generally, the optimization of gene expression is a combinatorial approach among these different elements in order to determine the optimal combination empirically.

Other enzymatic techniques have made it possible to synthesize molecules with high added value without resorting to microorganisms. These techniques, in which purified enzymes catalyze reactions with high specificity in vitro, make it possible to easily control the flows by controlling the reaction medium. The yields obtained can then be close to the theoretical yields, or even reach them. The direct use of enzymes, immobilized or not, thus overcomes some of the disadvantages of fermentation. However, these enzymes must be produced, purified and packaged beforehand, which makes the technique expensive. Even though the emergence of so-called cell-free systems has partly solved the problem of purifying proteins (enzymes) by synthesizing them concomitantly in the medium where the catalyzed reaction takes place, the cost of cofactors often essential to the reaction remains too high for a large-scale application of these techniques. Thus, all these approaches have serious disadvantages. Moreover, they are limited to the production of certain compounds. An alternative technique with the advantages of fermentation and biocatalysis, but without their respective disadvantages, and that can be adapted at will to the synthesis of a wide range of molecules of interest, could respond to current economic and environmental challenges.

To this end, the present invention proposes a technology using the biological machinery of microbial cells to produce different enzymes capable of catalyzing the reactions of a reaction scheme leading from a substrate to a compound of interest. The principle is to use the periplasmic space provided by bacteria (mainly Gram-negative bacteria) as a controlled reaction space, in which a given enzyme transforms a substrate and excretes the product formed into the culture medium. The product and any coproducts excreted by the first bacterium are then available to serve as a substrate for a bacterium expressing another enzyme, different from the enzyme produced by the first bacterium, thus forming a second product, and so on. This is called a cascade reaction using a consortium of at least 2 bacteria, each bacterium expressing an enzyme different from the enzyme produced by the other bacterium. From a bioprocess point of view, the increased accessibility to the substrate offered by the periplasm is a major advantage. The use of periplasmic secretion of enzymes offers improved accessibility to the substrate, compared to the so-called whole-cell bioprocess with cytoplasmic enzymes described above. The other advantage of expressing enzymes in the periplasm is to be able to control the pH and saline composition of the reaction medium. The pH of the periplasm and its ionic composition are equivalent to that of the surrounding medium, unlike the pH and ionic concentrations of the cytoplasm, which are regulated by the microorganism to maintain homeostasis (Wilks J C and Slonczewski J, 2007). However, to the knowledge of the applicant, no bioproduction process has ever been described using a consortium of at least two bacteria each expressing, in the periplasm, an enzyme different from the enzymes expressed by the other bacteria, in order to carry out several catalytic steps in cascade.

Firstly, it appeared, surprisingly, that bacteria modified to express different enzymes in the periplasm could be used in the same reaction medium, to each perform, in an orchestrated manner, at least one of the different reactions of a reaction scheme comprising several steps. Indeed, according to the invention, the different transformed strains brought into contact in the same reaction medium remain operational and perform their function in harmony.

Secondly, it has been unexpectedly demonstrated that the periplasmic wall of bacteria did not impede the circulation of products and reagents from one bacterium to another, so that not only are schemes comprising several reactions carried out in their entirety, but also the reaction kinetics are quite satisfactory to obtain the desired compound in just a few hours.

In addition, and despite the viscosity of the periplasm (gel-type state) linked to the presence of saccharides (Brass et al., 1986), which may have the effect of slowing down the enzymatic reactions, unlike the cytoplasm (liquid state) or the aqueous solutions used in vitro, the applicant unexpectedly observed that the resistance of the medium did not hinder carrying out reaction cascades according to the invention.

Finally, it is known that a major obstacle to the use of periplasm for bioproduction is the instability of cofactors in this compartment, yet cofactors are necessary in most metabolic pathways. For example, NAD (NAD+, its reduced state is NADH) is ubiquitous in metabolic reactions; it is the cofactor for more than 300 redox reactions (Zhou et al., 2011).

But thanks to the process according to the invention using a consortium of microorganisms, it is possible to recycle the cofactors necessary for enzymatic reactions, each of the enzymes being expressed in the periplasm of different cells. Cofactors, such as NAD(P)H, cannot cross the cytoplasmic membrane of bacterial cells, but they can diffuse through the periplasmic space that separates the outer membrane from the plasma membrane of bacteria. This allows the cofactors to move freely between the different cells of the consortium, allowing them to be efficiently recycled and reused for production.

Thus, sequential reaction schemes constituting complex biosynthesis pathways could be developed so as to be carried out by several bacteria, each expressing at least one enzyme in its periplasm. The periplasmic expression of enzymes, i.e., outside the cell proper, offers a controlled and simplified reaction medium from which the compounds of interest can be extracted without difficulty. Indeed, according to the process that is the subject of the present invention, enzymes are produced and transferred into the periplasm of the bacteria, a compartment in which they are confined but are available and catalytically operational with regard to the compounds solubilized in the medium. Insofar as the periplasmic compartment is easily accessible to solutes, the reactions take place therein in a protected environment that is much less complex than the cellular cytoplasm. This externalization of the reactions (outside the cellular cytoplasm) avoids the problems of flow redirection, toxicity and transport, specific to the cell. The reaction medium is, in fact, simplified and can be controlled to improve enzymatic reactions, in particular by the addition of cofactors and activators contributing to optimal activity. The final extraction phase of the molecule of interest is also facilitated. Since extraction can account for up to 70% of the cost of producing a compound by fermentation, this aspect is an essential asset.

A characteristic aspect of the proposed technique is that the biomass production process is decoupled from that of the production of the molecule of interest. The cell culture phase and the biochemical synthesis phase take place in separate reactors, with different media. As a result, the biomass in the reactor can be used for a new synthesis process or can be recovered after the synthesis of a compound and reused for a subsequent synthesis cycle. It is then possible to carry out multiple reactions from the same biomass. The costs of biomass production (cell multiplication of the different strains) are reduced accordingly, especially because of the significantly lower volumes of water to be treated. In doing so, production costs remain substantially those of microbial production costs. Another notable advantage of the dissociation of biomass production and the compound synthesis processes is that competition is avoided in the use of carbon sources. In a conventional fermentation process, the aim is to maximize the use of carbon for the production of the compound of interest in order to achieve a high yield, but this optimization may adversely affect cell growth (biomass production), and thus also hinder the production of the compound of interest. The balance between cell growth and production of the compound of interest is therefore critical. In contrast, in the inventive process, since biomass production is decoupled from molecule production, the carbon sources are distinct. It is therefore possible to optimize separately both the biomass growth and the production yield of the compound of interest. In addition, the applicant showed that it was possible to considerably increase the production of a product of interest by modulating the proportions of each biocatalyst (1 bacterium expressing 1 enzyme) in the consortium. This is a considerable advantage linked to the modular system of the process of the invention, compared to the previously described processes which would require complex genetic modifications of the same bacterium or other tools to increase production. In particular, it is known that the efficiency of signal peptides depends on the target protein to be produced. For example, a signal peptide that promotes high-level protein synthesis and secretion for one recombinant protein will often result in low-level protein synthesis and secretion for another (Karyolaimos et al., 2019). Since it is not possible to predict the performance of a signal peptide for a given recombinant protein, it is necessary to screen signal peptide libraries for one that promotes high-level protein synthesis and secretion. This approach is both time-consuming and costly. Thanks to the process according to the invention, it is possible to modulate the quantity of each biocatalyst (e.g. increase the quantity of biocatalyst to overcome the low efficiency of the signal peptide) in the reaction mixture for producing a product to maximize the efficiency of the reaction. It is thus possible to use a single signal peptide (e.g.: pelB), regardless of the protein to be produced, and increase the proportion of the biocatalyst in the case where the signal peptide does not have the expected efficiency on the production of the protein. This strategy makes it easy to improve the production of a product, which is advantageous for industrial use. The process that is the object of the invention, which will be described in detail below, can be described as a modular system, insofar as different bacteria, each transformed to express at least one enzyme in its periplasm, preferably a single enzyme, different from the enzymes expressed by the other bacteria of the system, can be cultured and stored, so as to provide, as needed, the effectors of a wide range of reactions to draw upon to construct a complete reaction scheme. We will talk about a library of biocatalysts, each comprising a bacterium expressing in its periplasm an enzyme, different from the enzymes expressed by the other bacteria of the biocatalyst library. The bacteria chosen to intervene in a defined scheme will be introduced into a single medium to act in concert in order to generate a product of interest. This approach, based on the combined implementation of cellular modules, has proved to be suitable for obtaining a wide variety of compounds, giving the inventive process a unique universal character. In particular, molecules with high added value, rare or difficult to access (poorly excreted, toxic, difficult to separate racemic isomers, etc.), have been obtained with high yields and a particularly favorable economic and environmental balance.

The use of synthetic microbial consortia according to the process of the invention thus has numerous advantages with regard to the bioproduction processes of the prior art: i) overcoming the high metabolic load conventionally borne by a single strain by dividing the load and allowing the pathways to be optimized in a modular manner, ii) allowing flexibility in balancing metabolic flows between individual modules by easily controlling the ratio of modified strains, iii) preventing the potential inhibition exerted by metabolic intermediates on sensitive enzymes, iv) finally, thanks to its modular nature, allowing the easy production of a variety of different chemical products by simply mixing different microbial strains. The process according to the invention thus has the advantage of avoiding the problems associated with the co-expression of several enzymes in a single cell and of overcoming the complexity of the regulation of gene expression. In addition, the quantities of each species of modified bacteria can be optimized for more efficient production of the product of interest.

More precisely, the present invention relates to a process for producing a compound of interest from an organic substrate, which comprises the steps consisting of:
a)—preparing a reaction mixture comprising, in a suitable medium,
   n bacteria, n being an integer at least equal to 2, each bacterium being genetically modified to express in its periplasmic space at least one enzyme E1, E2, . . . En, preferably a single enzyme, each of said n bacteria expressing an enzyme different from the other bacteria,
      an enzyme E1 being able to catalyze a first reaction R1 from said organic substrate to provide a first product P1, and, optionally, a first coproduct CoP1.
   each of said enzymes E2, . . . , En, being capable of catalyzing a reaction R2, . . . , Rn, from a product or a coproduct obtained by a reaction Rn−1, to provide, respectively, a product P2, . . . , Pn, and, optionally, a coproduct CoP2, . . . , CoPn,
said organic substrate, and
optionally, a cofactor of said organic substrate, a cofactor of said products obtained by a reaction Rn−1, called cofactors CoF1, CoF2, . . . , CoFn; a complementary substrate SC of a coproduct obtained by a reaction Rn−1,
b)—allowing the reaction mixture thus obtained to react, and
c)—separating the biomass from the supernatant and extracting therefrom said compound of interest consisting of one of the products P1, P2, . . . , Pn.

According to a particular and preferred embodiment, each bacterium is genetically modified to express in its periplasmic space a single enzyme E1, E2, . . . , En, each of said n bacteria expressing an enzyme different from the other bacteria.

The inventive process uses at least two genetically modified bacteria, each capable of secreting at least one given enzyme, different from the enzyme expressed by the other bacterium. Depending on the reaction scheme developed to produce the desired compound, it may comprise several bacteria, for example more than ten, and even several tens. This number is derived from the number of enzymes necessary to obtain a desired product from a given substrate (itself derived from the number of reaction steps), which will indicate the number of cellular modules to be used, without necessarily being equal. Indeed, the same bacterium can express more than one enzyme, in which case, the number of enzymes is greater than the number of bacteria. However, according to a particular and preferred embodiment, each bacterium will express a single enzyme, different from the enzymes expressed by the other bacteria. In particular, from 2 to 20 bacteria, especially from 3 to 15, or even from 4 to 12, may be used, each bacterium expressing an enzyme different from the enzymes expressed by the other bacteria.

Each bacterium is responsible for the production of at least one particular enzyme, preferably a single particular enzyme, so that if n enzymes are to be involved in the reaction scheme chosen, at most n bacteria will be involved in the process. The expression “module” or “cellular module” may be used to designate a genetically transformed bacterium and one of the enzymes R1, R2, . . . , Rn specifically expressed by this bacterium. To be clear, the integers from 1 to n may give an indication of the order of intervention of each enzyme in the chosen reaction scheme, but this is not a systematic rule, since some reaction schemes are linear, but others have branches. This is the case, in particular, when a module is used to regenerate a cofactor, which will be described in detail below. In any event, references n and n−1 or n and n+1 apply to directly consecutive reactions one downstream or upstream of the other, as well as to the enzymes and compounds involved in said reactions.

Thus, a reaction Rn−1 is defined as being directly upstream of a reaction Rn in the reaction scheme, so that the product Pn−1 and the coproduct CoPn−1, if any, resulting from the reaction Rn−1 are present in the medium and available to serve, respectively, as substrate, or, as applicable, as cofactor, for the reaction Rn directly downstream. There is an exception to this general principle concerning the first reaction of the reaction scheme, insofar as it is necessarily carried out from the chosen organic substrate that is transformed by the action of a first enzyme R1 to give a first product P1, and if appropriate a first coproduct CoP1. At each step, a product P1, P2, . . . , Pn is formed, as well as, optionally, a coproduct CoP1, CoP2, . . . , CoPn.

The reaction mixture prepared in the first step of the process according to the invention comprises, in addition to the cellular modules giving rise to the defined reaction scheme, a quantity of the organic substrate constituting the starting material to be converted in order to ultimately obtain the desired compound of interest. This initial organic substrate undergoes a first reaction catalyzed by the enzyme of the first module. Then, during each subsequent reaction step, the product synthesized during the reaction just preceding can act as a substrate to give a new product, and so on. In this linear reaction scheme, the product ultimately obtained will be the desired compound of interest.

Cofactors may be required for at least some of the reactions, which will result in the corresponding coproducts. The term “coproduct” here specifically designates a chemical species formed from a cofactor in the enzymatic reaction concerned. It should be noted that these coproducts are generally capable of acting as a cofactor in other reactions. Thus, it may be necessary to introduce into the reaction mixture, where appropriate, a cofactor of the organic substrate for the first reaction, or a cofactor of the product obtained by a reaction Rn−1 which will act as substrate in the following reaction R.

When in a reaction Rn−1, a substrate Sn−1 and a cofactor CoFn−1 react, a coproduct CoPn−1 is formed alongside a product Pn−1. A reaction scheme may provide that a coproduct CoPn−1 is itself consumed as a cofactor CoFn in the subsequent reaction Rn, alongside a complementary substrate. It is then particularly advantageous for the reaction Rn to be capable of reforming a cofactor CoFn−1. In this way, it is possible to regenerate the first cofactor, which eliminates the need to make a mass addition to the reaction mixture. However, it is advisable to add said complementary substrate in a sufficient quantity to the reaction mixture. In the present description, the expression “complementary substrate” designates a compound different from the initial organic substrate, which is used to react as a substrate alongside a cofactor. In this case, it will be necessary to introduce into the reaction mixture a substrate complementary to the cofactor in question, in this case the coproduct CoPn−1, which will react together in the following reaction R.

In summary, it will be understood that, according to the defined reaction scheme, one or more cofactors can intervene, as can one or more complementary substrates. This is why, in step a) of the process according to the invention, where appropriate, the various cofactors and complementary substrates are added to the reaction mixture.

The organic substrate may be any organic compound, the only reserve being that it is capable of constituting a substrate in an enzymatic reaction. It can therefore be of a very diverse nature, for example a linear or branched hydrocarbon and possibly substituted, such as an amine, an acid, a carbohydrate, an amino acid, or the like.

In step a) of the inventive process, the cellular modules and the organic substrate, optionally with the cofactors and/or complementary substrates involved, are introduced into a suitable medium, i.e., a medium that a person skilled in the art can prepare without difficulty according to the rules of the art known to them. Such a medium is an aqueous medium, generally to which minerals and a buffer have been added, such as those given below by way of example. It will be distinguished from the reaction medium proper, which is defined as the aqueous phase in which the reactions of the reaction scheme take place: This includes the medium in which the bacteria are immersed and their periplasmic space, since the solutes circulate easily from one to the other through the outer periplasmic wall. A simple buffer is used to facilitate the final purification of the compound obtained.

To be clear, steps a) and b) need not be strictly successive. In fact, the process can be carried out in continuous or semi-continuous mode, with ongoing or sequential addition of substrate and reagents. In addition, in certain cases, it is preferable not to add an additional substrate and/or a cofactor to the reaction mixture from the outset, but to do so within a certain period of time after the initiation of the reaction process. Similarly, one or more cellular modules may be introduced in an delayed manner. In other words, according to this particular protocol, an action of step a) of the process takes place while step b) has begun. This chronology is included in the context of the present invention.

It will be understood from the preceding that the process that is the object of the present invention offers multiple possibilities for designing reaction schemes adapted to the synthesis of various compounds of interest. It can be broken down in different ways which will now be presented in detail.

As already explained, the process according to the invention is based on an externalization of catalytic reactions into the periplasm of bacteria. Gram-negative diderm bacteria are primarily concerned, but any other organism possessing a periplasm can also be used. According to a preferred characteristic of the invention, the n genetically modified bacteria introduced into the reaction mixture are Gram-negative diderm bacteria, each selected from the Enterobacteriaceae, Alcaligenaceae, Vibrionaceae and Pseudomonadaceae family, for which the production of recombinant proteins has already been described (Stock et al., 1977; Eichmann et al., 2019; Karyolaimos et al., 2019). They may especially be enterobacteria belonging to the genera Salmonella, Yersinia or Escherichia. According to a more preferred embodiment, the bacteria are of the species Escherichia coli.

These bacteria are able to express in their periplasmic space one of the enzymes E1, E2, . . . , En, each bacterium expressing an enzyme different from the enzymes expressed by the other bacteria of the consortium. To do this, a genetic modification was induced to introduce into the cells a nucleic sequence coding for the enzyme concerned, associated with a nucleic sequence coding for a signal peptide. Each bacterium then produces a polypeptide in which two amino acid sequences are fused: one constituting the enzyme and the other the signal peptide. The fusion polypeptide is thus addressed into the periplasmic space, where the signal peptide is removed. The enzymes are then in the free state but confined in the periplasmic compartment, without having the capacity to pass through the outer wall. It has been verified that they remain in an active conformation and that they perfectly fulfill their catalytic function, each from substrates available from the outset or appearing as the reactions progress. Various signal peptides can be used in the context of the present invention, such as pelB, dsbA, EOX, lamB, MglB, MmAp, ompC, ompT, sufl, SfmC, STII, tolB, torA, torT, gIII, malE, ompA, phoA, or others chosen from those known to the person skilled in the art, who knows how to use them to allow periplasmic expression (Karyolaimos et al., 2019).

Thus, according to the invention, each of the bacteria is genetically modified to express at least one polypeptide, preferably a single polypeptide, comprising, respectively, one of said enzymes E1, E2, . . . , En, linked to a signal peptide addressing said polypeptide into the periplasmic space of said bacterium.

According to another embodiment, the enzymes or some of them may be anchored to the periplasmic wall. In this case, according to the invention, one or more of said n bacteria are genetically modified to express a polypeptide comprising, respectively, one of said enzymes E1, E2, . . . , En, linked to a membrane anchoring peptide including said signal peptide.

As already mentioned, a bacterium can be transformed so as to express in its periplasmic space more than one enzyme, i.e., two or more, responsible for catalyzing as many different reactions. These reactions may be successive reactions of the reaction scheme or non-successive reactions. Thus, according to one embodiment of the invention, at least one of said n bacteria is genetically modified to express in its periplasmic space at least two enzymes En-a and En-b capable of catalyzing at least two reactions Rn-a and Rn-b, so that said reactions RN-a and RN-b occur in the periplasmic space of said at least one bacterium. Note that, for the sake of clarity, this variant will not be developed for each embodiment described in the remainder of the description, but that it is expressly included in the present invention.

The transformation of the bacteria is carried out by insertion of the appropriate nucleic acid sequence using a suitable vector. One technique consists of penetrating into the bacterial cytoplasm a plasmid comprising a nucleic sequence capable of encoding the desired polypeptide. Another technique is based on the insertion of a nucleic acid sequence into the bacterial genome itself. These techniques and others are known in the field of cellular biotechnologies and will be implemented without difficulty by a person qualified in the field concerned.

Once transformed, each variant is cultured for biomass production and then used immediately or stored. It is convenient to produce a series of bacteria capable of expressing different enzymes, so as to have as many modules available for constructing reaction schemes that can be implemented by the process according to the invention. It is thus possible to draw on this cell library to custom prepare the desired reaction mixture in step a) of the process according to the invention.

Once the reaction mixture has been prepared in the appropriate medium, the reactions are allowed to proceed. This way of proceeding corresponds to a batch production mode. It should be noted that production in continuous or semi-continuous mode is also entirely possible as will be explained below. The reaction chain is interrupted spontaneously when the initial organic substrate is exhausted. The synthesized compound of interest is essentially excreted in solubilized form in the aqueous medium. According to the reaction scheme adopted, the compound will consist of one of the products P1, P2, . . . , Pn obtained in one of the reactions of the reaction scheme. After separation of the biomass, it can then be easily recovered in the supernatant, the composition of which is simple compared with that resulting from other previously known techniques.

According to a particular embodiment of the invention, the process can use two cellular modules, the first of which transforms the initial organic substrate, and the second of which uses the product of the first reaction to give a second product, which can be separated from the medium to obtain the desired compound. In this case, the reaction mixture comprises a bacterium expressing a first enzyme E1 capable of catalyzing the first reaction R1 from said organic substrate to provide a first product P1, and a bacterium expressing a second enzyme E2 capable of catalyzing a second reaction R2 from said first product P1, to form a second product P2, which is recovered in step c) as a compound of interest.

This protocol may be the beginning of a more extensive reaction scheme, in which case the second product may in turn be consumed as a substrate for a downstream reaction. In such an extended reaction scheme, the reaction mixture further comprises at least one bacterium expressing an enzyme E3, . . . , En, capable of catalyzing a reaction R3, . . . , Rn, from a product Pn−1 obtained by a reaction Rn−1, to provide a product Pn, which is recovered in step c) as a compound of interest or is consumed as a substrate of a reaction Rn+1.

When a cofactor is required to carry out the first reaction, this cofactor is introduced into the reaction medium in step a). The first cellular module is chosen to transform the initial organic substrate and this cofactor (called the first cofactor CoF1), and the second module will use either the product P1 of the first reaction to give a second product P2, or the coproduct CoP1 of the first reaction to give a second coproduct CoP2. The product P2 may be separated from the medium to obtain the desired compound or may be involved as a substrate in a downstream reaction of a larger reaction scheme. According to a particular protocol, the coproduct CoP2 may also be involved as a substrate for another reaction.

Thus, according to an embodiment of the process according to the invention, the reaction mixture comprises a cofactor CoF1 of said organic substrate, a bacterium expressing an enzyme E1 capable of catalyzing a first reaction R1 from said organic substrate and the cofactor CoF1 to form a first product P1 and a first coproduct CoP1; and a bacterium expressing a second enzyme E2 capable of catalyzing a second reaction R2 from the first product P1 to form a second product P2, or from the first coproduct CoP1 to form a second coproduct CoP2, at least one of the two being consumed in a third reaction, or recovered in step c) as a compound of interest. It will be understood that the expression “third reaction” here designates any one of the reactions which can take place downstream of the targeted reaction.

When the reaction scheme is more extensive, the above mechanism can be generalized. Indeed, each of the reactions Rn of a reaction scheme can use a cofactor CoFn reacting with the product Pn−1 generated by the upstream reaction Rn−1 under the effect of an enzyme En. A product Pn and a coproduct CoPn are then formed. The coproduct CoPn can then be used as the cofactor CoFn+1 of a downstream reaction Rn+1. For its part, the product Pn can be used as a substrate for an (other) downstream reaction Rn+1, unless it is the compound of interest sought. This protocol can be generalized to one or more of the modules involved.

This is why, according to one embodiment of the process that is the object of the invention, the reaction mixture comprises at least one cofactor CoFn, at least one bacterium expressing an enzyme capable of catalyzing a reaction Rn from the product Pn−1 of a reaction Rn−1 and said cofactor CoFn to form a product Pn and a coproduct CoPn, at least one of the two being consumed in a reaction Rn+1, or recovered in step c) as a compound of interest.

As has been seen, a reaction Rn of the reaction scheme can use as substrate a cofactor, which is generally a coproduct generated by an upstream reaction. In this case, another compound, called “complementary substrate”, should be present alongside it, which will give a complementary product resulting from the reaction Rn. This product may not be useful in the remainder of the reaction scheme and may accumulate in the medium. This is why, according to one characteristic of the process that is the object of the invention, the reaction mixture can also comprise at least one substrate SCn complementary to said at least one cofactor CoFn.

In the process according to the invention, said at least one cofactor CoFn can be introduced entirely or partially into the reaction mixture in step a), or during step b). In fact, it is sometimes preferable to add a cofactor CoFn that is not involved in the first steps of the reaction scheme only when the product Pn that is to react with said cofactor CoFn is present in the medium in a significant amount. The reaction kinetics are thus taken into account in order to optimize the production of the compound of interest.

Moreover, when a cofactor is necessary for a first reaction, it is particularly advantageous for a second reaction (downstream reaction) to allow its regeneration. Indeed, cofactors are expensive compounds that directly impact production costs. A reaction scheme has been devised to meet this objective, in which the second reaction consumes the coproduct from the first reaction as a cofactor in the second reaction, to reform the initial cofactor. In doing so, it is advisable to provide a complementary substrate for the second reaction, which will be chosen so that it has a moderate cost. At the end of the second reaction, a second product is formed which may or may not be of interest, but, above all, the cofactor of the first reaction is regenerated.

Thus, in a particularly advantageous embodiment of the process according to the present invention, said at least one cofactor CoFn is formed at least in part in the reaction mixture by a reaction Rn−1 catalyzed by an enzyme En-1 capable of forming a product Pn−1 and a coproduct CoPn−1, said coproduct CoPn−1 being identical to the cofactor CoFn.

However, it is preferable to add a certain amount of this cofactor to the medium, in order to initiate the process until regeneration of the cofactor is sufficient to ensure a continuous supply of said cofactor. Now, it has become apparent that a minimal quantity is sufficient to initiate the reaction cycle. The quantity of cofactor that it is necessary to introduce into the reaction mixture for the reaction concerned to proceed completely is then significantly reduced with respect to the quantity of the corresponding substrate with which it must react. It may be at least 20 times lower, but preferably at least 100 times lower, more preferably at least 500 times lower, and even better at least 1000 times lower. It should be noted that certain cofactors synthesized in the cellular cytoplasm, such as ATP, for example, may be present in the periplasmic space in a small, but sufficient quantity.

Thus, in this context, according to the invention, the quantity of the first cofactor CoF1 introduced into the reaction mixture in step a) corresponds to a molar concentration at least 20 times lower than the initial molar concentration of said organic substrate.

As indicated above, the initial organic substrate may be of various natures, including any kind of carbon-containing molecules. The process has, in particular, demonstrated its effectiveness in transforming common carbohydrates (sugars) such as glucose, fructose, glycerol, or others, into compounds of interest such as rare sugars, especially phosphorylated sugars, particular isomers of rare sugars, dihydroxyacetone and other molecules sought to give access to important synthetic routes. Other organic compounds may constitute the starting substrate, including aldehydes, alcohols, organic acids, carbamates, hydrocarbons and amino acids. Examples include glyoxal providing glycolaldehyde, or L-phenylalanine, the starting point for the production of 2-phenylethanol. By extension, the organic substrate may also be a compound comprising a single carbon atom, such as carbon dioxide.

Thus, according to one characteristic of the process that is the object of the present invention, the organic substrate of the first reaction is chosen from carbohydrates, aldehydes, alcohols, organic acids, carbamates, hydrocarbons, amino acids and carbon dioxide.

Numerous enzymes can be produced by the various transformed bacteria used in the inventive process. A person skilled in the art knows how to identify those suitable for the development of a reaction scheme leading from an organic substrate to a given compound of interest. They can select them from those that are listed and described or that will be identified in the future.

Thus, in accordance with a preferred characteristic of the process according to the invention, the enzymes E1, E2, . . . , En are each chosen from kinases, dehydrogenases, phosphatases, reductases, isomerases and transferases. The kinases may especially be a glycerol kinase, a glucokinase, a fructokinase, a xylokinase, an acetate kinase, or a phosphofructokinase. The dehydrogenases are, for example, glycerol dehydrogenase or phosphite dehydrogenase. Reductases such as NADH reductase or glyoxal reductase can be used. The isomerases may be chosen from xylose isomerase, xylulose isomerase, ribulose phosphate-3-epimerase, ribose-5-phosphate isomerase, while transketolase or transaldolase may be chosen as transferase. Various phosphatases are also known.

Moreover, multiple cofactors may be involved in one or another of the reactions of the reaction scheme. It should be noted that as the cofactors forming the coproducts that can, in turn, play the role of cofactor in an upstream or downstream reaction, the molecules concerned belong to the two categories. Thus, preferably, in the process according to the invention, at least one of the enzymes E1, E2, . . . , En catalyzes a reaction providing one of the coproducts CoP1, CoP2, . . . , CoPn, chosen from ATP, ADP, AMP, UTP, UDP, UMP, NAD+, NADH, NADP+, NADPH, FAD, FADH₂, coenzyme A, or catalyzes a reaction using one of them as cofactor.

It has been seen that at the end of step c) of the inventive process, the biomass is separated from the supernatant from which the compound of interest is extracted. However the work carried out has revealed an unexpected advantage of the inventive process. Indeed, it has appeared that this biomass, containing the cells of the n bacteria introduced in step a), can be reused as it is in a new reaction cycle, with a yield of the same level and sometimes even greater than that observed during the first reaction cycle. This results in a significant savings in terms of time, number of operations to be carried out, consumption of ingredients and water and finally, in economic terms. This advantage results from the principle of the invention according to which the cell culture phase is dissociated from the biochemical synthesis phase: They take place in separate reactors, with different media.

The supernatant can be separated from the biomass by gravity flow, and in this case it is possible to directly reuse the biomass remaining in the reactor, adding only fresh substrate and, optionally, the other necessary ingredients.

Thus, in a particularly advantageous manner, after step c) of the process that is the object of the present invention, the biomass separated from the supernatant is reused to prepare a new reaction mixture according to step a), with or without an intermediate preservation step.

The process may be implemented in the context of discontinuous batch cultures as described above, or by discontinuous flow (fed batch type). It can also be conducted in semi-continuous mode or with continuous supply, according to the known rules of the art. In this case, steps a) and b) do not take place sequentially, but more or less simultaneously.

This is why, in a variant embodiment of the process according to the invention, steps a) and b) are carried out by adding to the reaction mixture containing said n bacteria, continuously or at intervals of time,
said organic substrate, and
optionally, a cofactor of said organic substrate, a cofactor of said products obtained by a reaction Rn−1, called cofactors CoF1, CoF2, . . . , CoFn; a complementary substrate SC of a coproduct obtained by a reaction Rn−1, and by allowing the reaction mixture thus obtained to react continuously or semi-continuously.

It emerges from the preceding that the approach adopted by the process for producing compounds of interest as has just been described has numerous advantages responding to the difficulties encountered with conventional techniques and is very general in scope. The externalization of the reactions in fact makes it possible to use the biochemical machinery of the cell to supply enzymatic effectors of reactions taking place in the space delimited by the periplasm, while allowing the reagents and products involved in the reactions to circulate. The combinable module design allows complete original reaction schemes to be developed and implemented quickly. The synthesis of a given product of interest is thus facilitated, as is its final purification, since the aqueous bioproduction medium is very simple.

It should be added that since the process relies on the production of reusable biomass, it is much cheaper than the known enzymatic processes (when they exist). It is therefore possible to industrially produce molecules of all kinds, including those that are complex or difficult to access.

It is thus understood that the process that is the object of the invention represents an unprecedented alternative to current production methods and offers a powerful tool for accelerating the development of new products.

According to a second aspect, the present invention concerns a reaction mixture for the production of a compound of interest from an organic substrate. Such a reaction mixture is essentially identical to the one prepared in step a) of the production process described above.

Thus, the present invention relates to a reaction mixture for the production of a compound of interest from an organic substrate, containing, in a suitable medium:
n bacteria, n being an integer at least equal to 2, each bacterium being genetically modified to express in its periplasmic space at least one enzyme E1, E2, . . . En, preferably a single enzyme, each of said n bacteria expressing an enzyme different from the other bacteria,
   an enzyme E1 being able to catalyze a first reaction R1 from said organic substrate to provide a first product P1, and, optionally, a first coproduct CoP1,
   each of said enzymes E2, . . . , En, being capable of catalyzing a reaction R2, . . . , Rn, from a product or a coproduct obtained by a reaction Rn−1, to provide, respectively, a product P2, . . . , Pn, and optionally a coproduct CoP2, . . . , CoPn,
said organic substrate, and
optionally, a cofactor of said organic substrate, a cofactor of said products obtained by a reaction Rn−1, called cofactors CoF1, CoF2, . . . , CoFn; a complementary substrate SC of a coproduct obtained by a reaction Rn−1.

According to a particular and preferred embodiment, each bacterium is genetically modified to express in its periplasmic space a single enzyme E1, E2, . . . , En, each of said n bacteria expressing an enzyme different from the other bacteria.

The reaction mixture is therefore a mixed composition comprising cells in suspension of at least two bacteria, and various soluble reagents, including the organic substrate to be transformed in order to obtain the compound of interest. Reference may be made to the preceding description in order to define the various embodiments of this mixture. According to a particular embodiment, the reaction mixture comprises at least 3 bacteria, especially at least 4 bacteria, or even at least 5 bacteria, and in particular at least 6 bacteria, each bacterium being genetically modified to express an enzyme different from the enzymes expressed by the other bacteria.

Finally, according to a third aspect of the present invention, the use of a reaction mixture as described above is claimed to produce a compound of interest chosen from carbohydrates, aldehydes, alcohols, organic acids, carbamates, hydrocarbons and amino acids. By way of example, a reaction mixture comprising a carbohydrate can be used to produce a phosphorylated sugar or dihydroxyacetone (DHA), or a reaction mixture comprising an amino acid can be used to produce a nitrogenous organic compound.

The present invention will be better understood and details thereof will become apparent in light of the description which will be given of various embodiments, with reference to the attached figures.

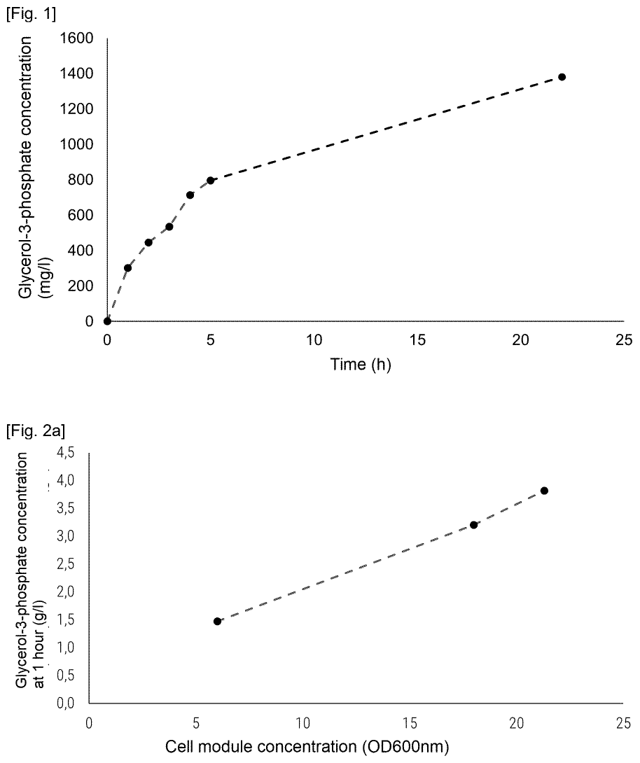

FIG. 1: Production of glycerol-3-phosphate over time from glycerol by two cellular modules (BL21-Glpk and BL21-AckA).

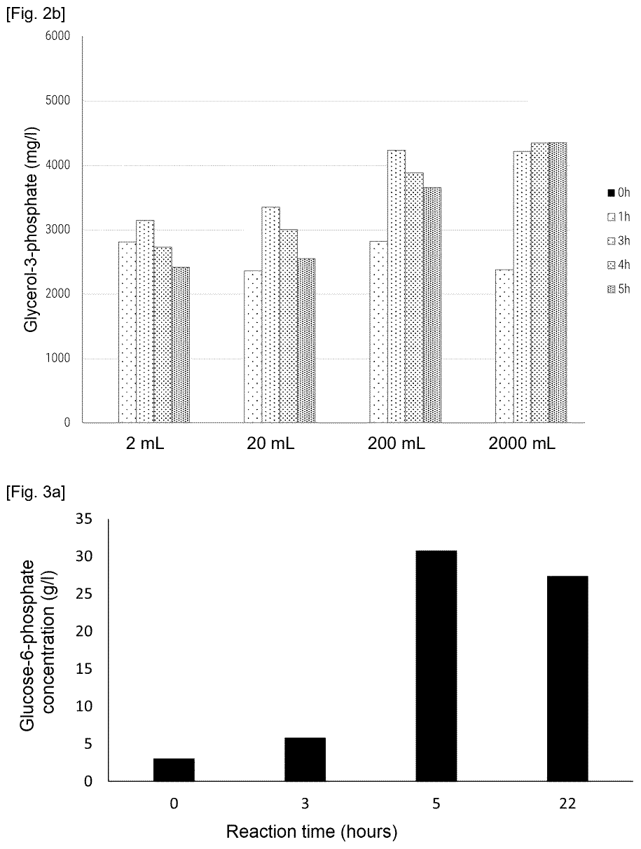

FIG. 2a: Effect of the concentration of cellular modules (BL21-Glpk and BL21-AckA) on the production of glycerol-3-phosphate.

FIG. 2b: Effect of the reaction volume-production of glycerol-3-phosphate in volumes of 2 ml, 20 ml, 200 ml and 2000 ml.

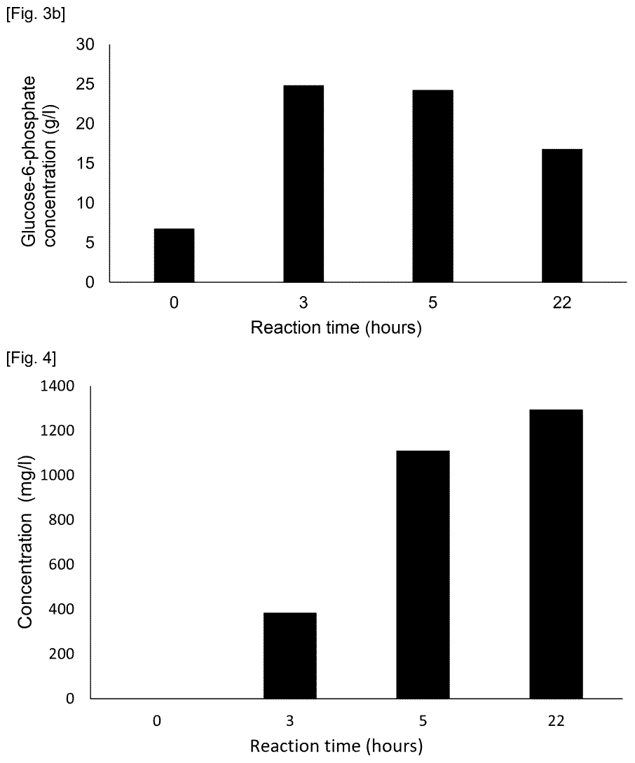

FIG. 3a: Production of D-glucose-6-phosphate from D-glucose by two cellular modules BL21-Glk and BL21-AckA.

FIG. 3b: Production of D-glucose-6-phosphate from D-glucose by two cellular modules BL21-nlpA-Glk and BL21-nlpA-AckA.

FIG. 4: Production of fructose-1-phosphate from D-fructose by two cell modules BL21-KhkC and BL21-AckA.

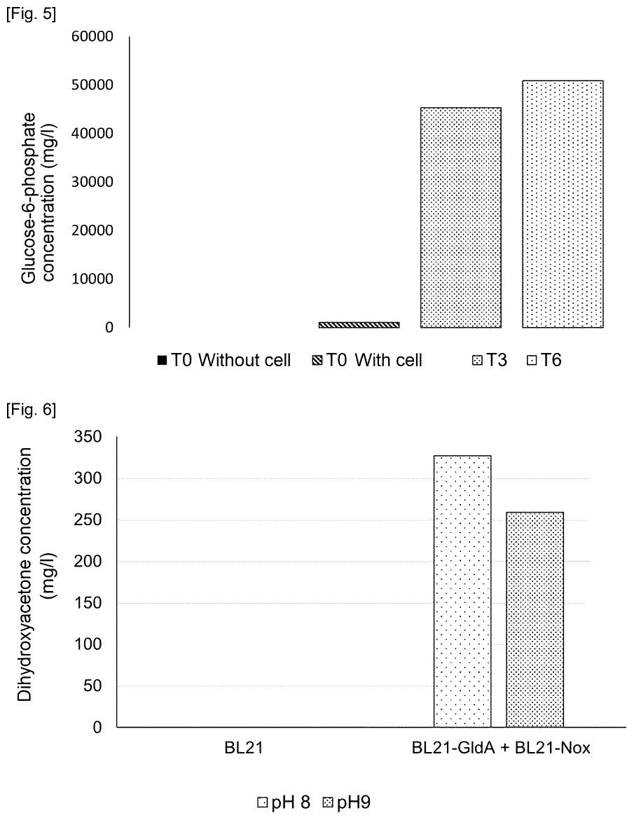

FIG. 5: Production of glucose-6-phosphate with reuse of the BL21-Glk and BL21-AckA cell modules.

FIG. 6: Production of dihydroxyacetone from glycerol by the BL21-GldA and BL21-Nox modules.

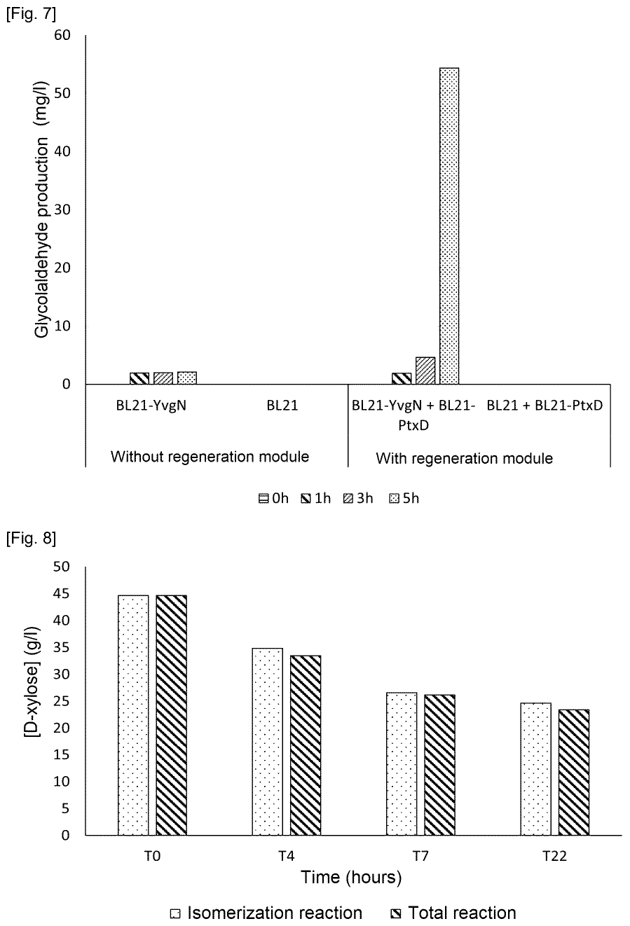

FIG. 7: Production of glycolaldehyde from glyoxal by the BL21-YvgN module, with or without the BL21-PtxD regeneration module.

FIG. 8: Consumption of D-xylose by the isomerization reaction alone (BL21-XylA) and by the total reaction (BL21-XylA, BL21-XylB, BL21-AckA, BL21-AphA).

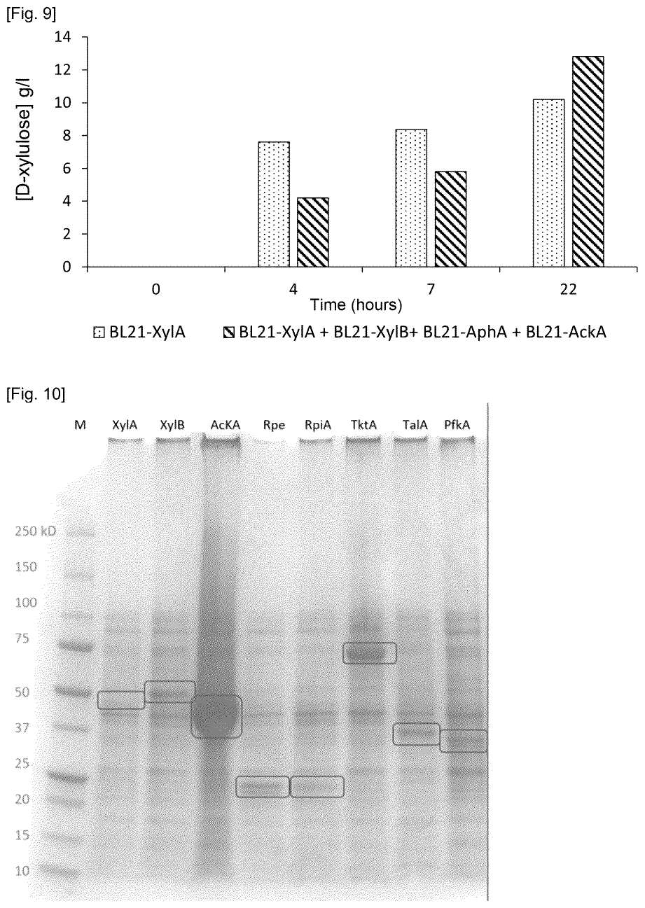

FIG. 9: Production of D-xylulose from D-xylose by the isomerization reaction alone (BL21-XylA) and by the total reaction (BL21-XylA, BL21-XylB, BL21-AckA, BL21-AphA).

FIG. 10: SDS-PAGE gel migration of periplasmic fractions of XylA, XylB, AckA, Rpe, RpiA, TktA, Tal, PfkA modules and the control BL21 WT.

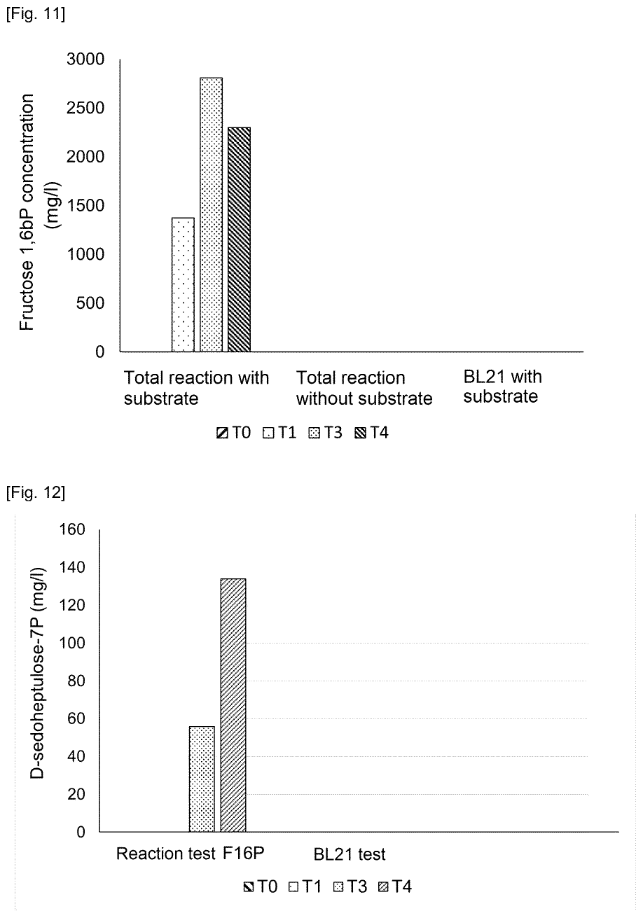

FIG. 11: Production of D-fructose-1,6-bisphosphate from D-xylose using eight cellular modules.

FIG. 12: Production of the intermediate D-sedoheptulose-7-phosphate during the production of D-fructose-1,6-bisphosphate with eight modules.

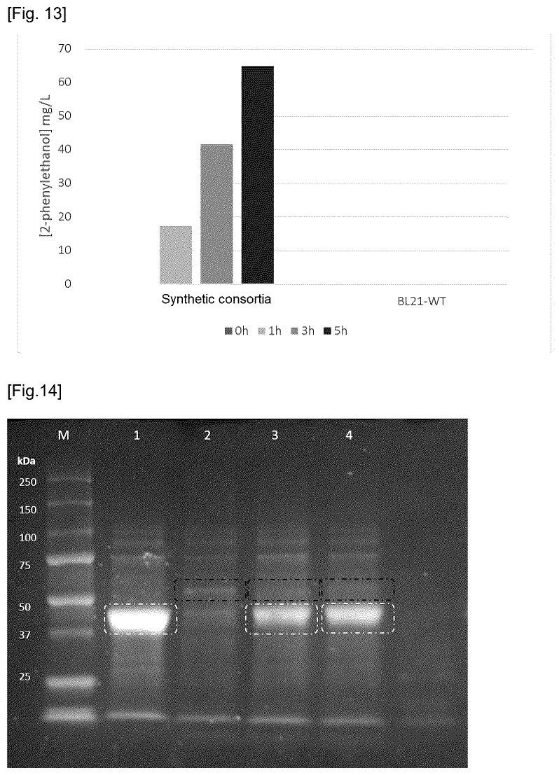

FIG. 13. Monitoring the concentration of 2-phenylethanol by HPLC in a synthetic consortia composed of strains BL21-ARO8, BL21-ARO10, BL21-ADH5 and BL21-PtxD and in the control BL21-WT in the presence of 10 mM L-phenylalanine.

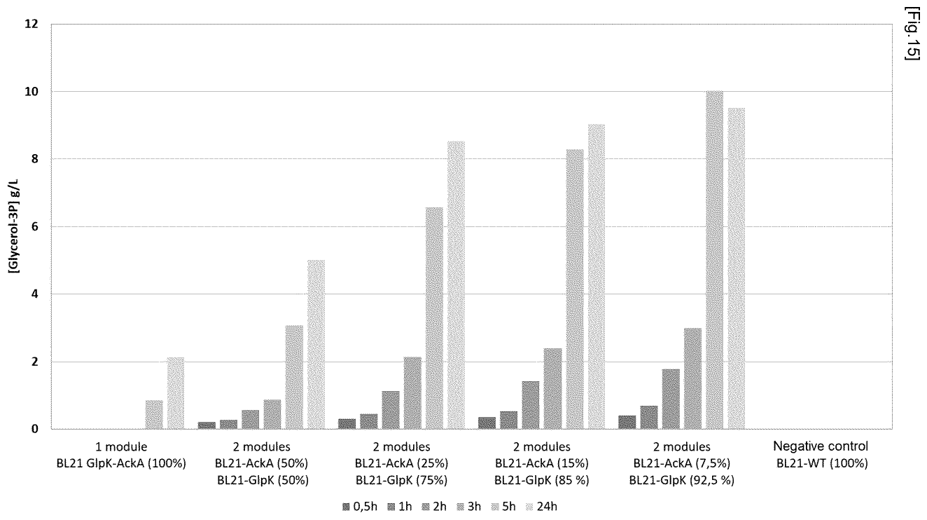

FIG. 14. SDS-PAGE gel of the periplasmic fractions of the BL21-AckA, BL21-GlpK, BL21-AckA-GlpK biocatalysts and of the negative control BL21-W.

M: Precision Plus Protein™ All Blue Marker, BIO-RAD®; 1: BL21-AckA (2 OD_{600 nm}/ml); 2: BL21-GlpK (2 OD_{600 nm}/ml); 3: BL21-GlpK-AckA (2 OD_{600 nm}/ml), 4: BL21-GlpK (1 OD_{600 nm}/ml), BL21-GlpK (1 OD_{600 nm}/ml), 5:BL21-WT (2 OD_{600 nm}/ml); black dashed frame: GlpK protein of 56 kDa; white dashed frame: AckA protein of 43 kDa.

FIG. 15. Production of glycerol-3-phosphate from glycerol by the strain BL21-GlpK-AckA, the synthetic consortia composed of the 2 modules BL21-AckA and BL21-GlpK supplied in variable proportions and the control BL21-WT.

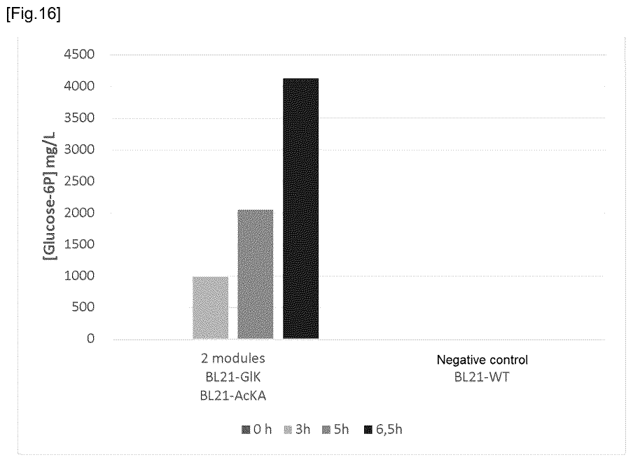

FIG. 16. Production of glucose-6-phosphate in a synthetic consortia composed of 2 BL21-Glk, BL21-AckA modules and in the BL21-WT control in the presence of glucose and without the addition of ATP.

### MATERIALS AND METHODS

### 1—Plasmids

Plasmid pET22b (ori pBR322, ampicillin, T7 promoter, pelB) and plasmid pET26b (ori pBR322, kanamycin, T7 promoter, pelB) are commercial plasmids carrying the pelB signal sequence at the N-terminal and a His-tag sequence at the C-terminal. They were used for the expression of a gene of interest coding for an enzyme that will be transported by PelB fusion peptide into the periplasm of the cell.
Plasmid pACT3 (ori p15A, chloramphenicol, Ptac promoter) was used to anchor proteins in the periplasm. The plasmid was modified to have the N-terminal leader sequence of the lipoprotein NlpA.
Plasmid pETDuet-1 (ori pBR322, ampicillin, T7 promoter) is designed for the co-expression of two genes of interest. The vector carries two cloning sites, each preceded by a T7 promoter, a lac operator and a Shine-Dalgarno sequence. Plasmid pETDuet-1 was modified here by integrating the PelB signal sequence after the two ribosome binding sites. The expression cassettes (T7-RBS-pelB) are identical to that of pET22b. The genes integrated into this modified plasmid allow the co-expression of two free enzymes in the periplasm under the control of two T7 promoters inducible with IPTG.

The genes are amplified from the genomic DNA of Escherichia coli MG1655 by a technique known per se, for example by polymerase chain reaction (PCR) using the appropriate primers, then inserted into plasmids pET22b, pET26b, pACT3 or pETDuet-1 previously linearized with the NEBuilder® kit by homologous recombination. If specified, synthetic genes were directly ordered from GenScript® and directly cloned by them into plasmid pET22b or pET26b.

The primers used for gene amplification are of a generally known type. The person skilled in the art knows how to choose and use them in the context of PCR techniques. According to a particular embodiment, the primers for the amplification of each of the genes are prepared using the kit and the instructions of the manufacturer NEB “NEBuilder HiFi DNA Assembly Master Mix & NEBuilder HiFi DNA Assembly Cloning Kit E2621 E5520” (https://www.neb.com/-/media/nebus/files/manuals/manuale2621_e5520.pdf?rev=67d09a762b2c47ed8df3a0bd1cb59a1 b&hash=02CF464149634283F5633CA1 DE4D2FDF). In particular and according to the manufacturer's recommendations, the PCR primers to be used in the assembly of HiFi DNA must have two sequence components: i) an overlapping sequence, necessary for the assembly of adjacent fragments; ii) a sequence specific to the gene, required for priming of the template during PCR. To obtain an effective assembly of PCR fragments in a vector, it is suggested to use an overlap of 15 to 30 nucleotides with a Tm equal to or greater than 48° C. (assuming that the A-T pair=2° C. and the G-C pair=4° C.). Examples of the above-mentioned plasmids include the following primers: GLPK_pET22b; Khk-C_pET22b; ackA_pET26b; gldA_pET22b; XylA_pET22b; XylB_pET22b; GLK_pET22b; GLK_pET22b; pET22B_rpe; pET22B_rpiA; pET22b_tktA; pET22b_tal; pET22b_pfkA; nlpA_glk; pACT3_glk; nlpA_ackA; pACT3_ackA; pETDuet_glpK_ack; pET22b_ARO8; pET22b_ARO10; pET22b_ADH5.

### 2—Construction of the Strains

Strains BL21(DE3) or BL21(DE3) gold were chosen for the transformation of plasmids pET22b or pET26b (depending on the resistance cassette) in order to be able to use T7 polymerase. The transformations were carried out according to the TSS protocol described by Chung and Miller (Chung et al., 1993). The last two strains were constructed so that the enzyme was anchored in the periplasm, all the others so that it was free. The strains used are given in Table 1.

TABLE 1
Escherichia coli strains used

| Gene | | | | | | |
|---|---|---|---|---|---|---|
| overexpressed | Gene ID | | Module | | | Strain: |
| (Organism) | (NCBI) | GenBank | name | Plasmid | Resistance | Genotype |
| xylA (E. coli) | 948141 | X0772.1 | BL21-XylA | pET22b - | Amp | BL21 (DE3): |
| | | | | xylA | | E. coli str. B |
| xylA (E. coli) | 948133 | X04691.1 | BL21-XylB | pET22b - | | F- ompT gal |
| | | | | xylB | | dcm Ion |
| AckA (E. coli) | 946775 | M22956.1 | BL21-AckA | pET26b- | Kan | hsdSB(rB- |
| | | | | AckA | | mB-) λ(DE3 |
| rpe (E. coli) | 947896 | Z19601 | BL21-Rpe | pET22b - | Amp | [lacl lacUV5- |
| | | | | rpe | | T7p07 ind1 |
| rpiA (E. coli) | 947407 | X73026 | BL21-RpiA | pET22b - | | sam7 nin5]) |
| | | | | rpiA | | |
| tktA (E. coli) | 947420 | X68025 | BL21-TktA | pET22b - | | |
| | | | | tktA | | |
| talA (E. coli) | 947006 | D13159 | BL21-TalA | pET22b - | | |
| | | | | talA | | |
| pfkA (E. coli) | 948412 | X02519 | BL21-PfkA | pET22b - | | |
| | | | | pfkA | | |
| Nox | | CP002461.1 | BL21-Nox | pET22b - | | |
| (Lactobacillus | | | | nox | | |
| sanfransiscensis) | | | | | | |
| gldA (E. coli) | 948440 | U00006 | BL21-GldA | pET22b - | | |
| | | | | gldA | | |
| yvgN (B. subtilis) | 936001 | AJ223976 | BL21- | pET22b - | | |
| | | | YvgN | yvgn | | |
| glk (E. coli) | 946858 | U22490 | BL21-Glk | pET22b - | | |
| | | | | glk | | |
| glpK (E. coli) | 948423 | M18393 | BL21-GlpK | pET22b - | | |
| | | | | glpk | | |
| khkC (H. | 3795 | X78678 | BL21- | pET22b - | | |
| sapiens) | | | KhkC | khkc | | |
| ptxD | 69882623 | AF061070 | BL21-PtxD | pET22b - | | |
| (Pseudomonas | | | | ptxD | | |
| stutzeri) | | | | | | |
| aphA (E. coli) | 948562 | U51210 | BL21- | pET22b- | | |
| | | | AphA | apha | | |
| / | | | BL21-WT | pET22b- | | |
| | | | | MSC | | |
| glpK (E. coli) | 948423-946775 | M18393-M22956.1 | BL21- | pETDuet1- | | |
| AckA (E. coli) | | | GlpK-AckA | pelB-glpK- | | |
| | | | | pelB-ackA | | |
| ARO8 (S. | 852672 | Y13624 | BL21- | pET22b- | | |
| cerevisiae) | | | ARO8 | ARO8 | | |
| ARO10 (S. | 851987 | U28373 | BL21- | pET22b- | | |
| cerevisiae) | | | ARO10 | ARO10 | | |
| ADH5 (S. | 852442 | Z36014 | BL21- | pET22b- | | |
| cerevisiae) | | | ADH5 | ADH5 | | |
| glK (E. coli) | 946858 | U22490 | BL21-nlpA- | pACT3- | Chm | |
| | | | Glk | nlpA-glk | | |
| ackA (E. coli) | 946775 | M22956.1 | BL21-nlpA- | pACT3- | | |
| | | | AckA | nlpA-AckA | | |

### 3—Culture Media and Conditions

### Induced Whole Cell Production

The strains are taken up from a glycerol stock stored at −80° C., in 5 ml of TB medium buffered to pH 7 with the appropriate antibiotic and placed at 37° C. overnight. The composition of the TB medium is given in Table 2.

TABLE 2
composition of TB medium buffered to pH 7

| Compound | Quantity | Unit |
|---|---|---|
| Yeast extract | 24 | g/l |
| Tryptone | 20 | g/l |
| Glycerol | 5 | g/l |
| Kh₂PO₄ | 0.017 | M |
| Na₂HPO₄ | 0.072 | M |

The next day, the preculture is diluted to 1/100th in 50 ml of fresh TB medium and the culture is stirred at 200 rpm at 37° C. When the optical density at 600 nm (OD 600 nm) reaches a value between 0.4 and 0.6, the cells are induced with 0.1 mM IPTG. They are then placed at 20° C. with stirring at 200 rpm, for approximately 18 hours.

### External Production of Target Molecules

The induced whole cells expressing one or more enzymes in their periplasm (or modules) are centrifuged at 4500 rpm for 10 minutes and resuspended in sterile water in order to reach a defined quantity of cells expressed in ODU. The modules carrying the enzymes, chosen according to the requirements of a predefined reaction scheme, are introduced together into the reaction mixture. This comprises a buffer to stabilize the pH, if necessary activators and cofactors, the substrate and a known quantity of each cell module. The composition is deliberately as simple as possible in order to ensure optimal enzymatic activity and a minimized production cost. The substrates and cofactors were purchased from Sigma® or Carbosynth®, with the exception of the acetyl phosphate used for ATP regeneration, which was synthesized according to the method described in Crans et al., 1983, from phosphoric acid (85%, 2 mol) and ethyl acetate (2 mol), two low-cost compounds.

### 4—Quantification and Analysis of Extracellular Products

The substrate and product concentrations are monitored by high pressure ion chromatography (HPIC) or by high performance liquid chromatography (HPLC). Different detection methods were used depending on the target molecules.

### HPIC Analysis

The samples are centrifuged at 14,800 rpm for 2 minutes and then filtered at 0.2 μm. The samples to be analyzed are diluted to 1/100th in milli-Q water in flasks equipped with a pre-pierced stopper and placed at 15° C. in an autosampler.

### Amperometric Detector (HPIC-PAD)

The analysis of certain substrates and products is done by ion chromatography (Dionex™ ICS-6000 ion chromatography system) coupled with pulsed amperometric detection or PAD (Dionex™ ICS-6000 Electrochemical Detector). The molecules are separated on a Dionex™ Carbopac™ PA1 column (2×250 mm) preceded by a guard column of the same type (2×50 mm) at 25° C.

Two elution methods were used. The method used for the determination of sugars (xylose, glucose, fructose), organophosphorus compounds (glucose-6P, fructose-1P, fructose-1,6bP, glycerol-3P), methylglyoxal and aldehydes such as glycolaldehyde is as follows: The NaOH concentration is maintained at 100 mM from 0 to 30 min while the sodium acetate concentration (NaOAc) varies gradually. From 0 to 2 min, 0 mM NaOAc; from 2 min to 15 min increase from 0 to 500 mM NaOAc; from 15 min to 23 min concentration maintained at 500 mM NaOAc; from 23 min to 23.1 min decrease from 500 mM to 0 mM NaOAc; from 23.1 min to 30 min maintain at 0 mM NaOAc.

### Conductivity Detector (HPIC-CD)

Anions (organophosphorus compounds, phosphate, chloride, sulfate, organic acids, etc.) are analyzed by ion chromatography (Dionex™ CS-6000 ion chromatography system) equipped with a conductivity meter (Dionex™ CS-6000 CD Conductivity Detector). The molecules are separated on a Dionex™ IonPac™ AS11-HC column (2×250 mm) preceded by a guard column of the same type (2×50 mm) at 25° C.

The concentration gradients are created with the eluent generator EGC 500 KOH at a flow rate of 0.35 ml/min. The following gradient was used to best separate the intermediate compounds from the reaction scheme presented in Example 7. Before each injection, conditioning for 7 min with 2 mM NaOH is carried out. The first step of the gradient is an isocratic elution at a NaOH concentration of 10 mM for 3 min followed by an increase in NaOH concentration in several steps: 1) increase from 10 mM to 50 mM in 9 min; 2) increase from 50 mM to 100 mM in 7 min; 3) maintain at 50 mM for 3 min; 4) decrease in NaOH concentration to 10 mM; 5) maintain at 10 mM for 4 min. At the column outlet, an ADRS 2 mm suppressor (87 mA) is used to improve detection. For the other examples, the NaOH concentration gradient presented below was used for the determination of the anionic metabolites. The first step is an isocratic elution at 2 mM NaOH concentration for 4 min followed by a multi-step increase in NaOH concentration: 1) increase from 2 mM to 100 mM in 11 min; 2) maintain at 100 mM for 10 min; 3) decrease to 2 mM and maintain for 1 min.

### UHPLC-UV/RI Analyses

Sugars and organic acids are analyzed by UHPLC (Dionex Ultimate™ 3000) equipped with a refractometer (Shodex™ RI-101) and a UV detector (Dionex UltiMate™ 3000 Diode Array Detectors 3000(RS)). The molecules are separated on a Phenomenex™ ROA-Organic Acid H+ column (8%) (300×7.8 mm) with a guard column of the same type (50×7.8 mm). Elution is done isocratically (5 mM H₂SO₄) at a flow rate of 0.5 ml/min for 35 minutes. The samples to be analyzed are filtered at 0.2 μm before injection.

### HPLC-Mass Spectrometry Analyses

The presence of fructose-1-phosphate is detected by UHPLC (Dionex Ultimate™ 3000) equipped with a mass spectrometer (Thermo Fisher Scientific™). The molecules are separated on an ACQUITY UPLC BEH Amide Column (130 Å, 1.7 μm, 2.1×30 mm) preceded by a guard column (ACQUITY BEH Shield RP18 1.7 μm VANGUARD). Mobile phase A is composed of ultrapure water and methylphosphonic acid (1.8 mM), and organic mobile phase B is composed of acetonitrile and formic acid (1.3 mM). The concentration gradient used is as follows at a flow rate of 0.35 ml/min: 1 min isocratically at 90% organic mobile phase, followed by decreases in the organic mobile phase fraction in several steps: 82% at 3 min, 78% at 6.5 min and 50% at 8 min. This composition is kept constant for 10 min (50% of organic phase) followed by an equilibration time of 7 min.

Glycerol-3-phosphate C13 (G3P-C13) is used as the internal standard. An elution gradient is created. The injected mixture is prepared in inserts containing 100 μl of the undiluted filtered sample, 100 μl of eluent (acetonitrile+1.3 mM formic acid) and 10 μl of internal standard (G3P-C13). The final dilution of the sample is 2.1. This dilution factor is taken into account when processing results with Chromeleon™ software.

### 5—Extraction of Proteins by Osmotic Shock

The cells are cultured in 2 ml of LB medium overnight, at 37° C. and with stirring at 200 rpm. The cell suspension is transferred into an Eppendorf tube and centrifuged (2 ml; 5 min; 14,000 g; 4° C.). The pellet is resuspended in 500 μl of buffer 1 and incubated for 20 min on ice. The tubes are inverted regularly to avoid sedimentation. The cell mixture is centrifuged (15 min; 14,000 g; 4° C.). The supernatant is discarded and the cell pellet is resuspended in 125 μl of buffer 2. After further incubation for 10 to 20 minutes on ice, with regular inversion, the cells are centrifuged (15 min; 14,000 g; 4° C.). The supernatant containing the periplasmic fraction is recovered, then denatured for 10 min at 95° C. and deposited on SDS-PAGE gel (45 min, 180 V, 400 mA) to verify the presence of the enzyme and its molecular weight. The size marker used is Precision Plus Protein™ All Blue Standards #1610373EDU. The composition of the buffers is given in Table 3.

TABLE 3
Composition of buffers 1 and 2

| Buffer 1 | Buffer 2 |
|---|---|
| 0.2M Tris - HCl | 0.01M Tris HCl |
| (pH 8.0) | (pH 8.0) |
| 200 g/l sucrose | 0.005 g/l MgSO₄ |
| 0.1M EDTA | 0.2% SDS |
| | 1% Triton X-100 |

### Example 1: Production of Phosphorylated Compounds

Phosphorylation is a reaction that allows cells to sequester essential metabolites in the cytoplasm and prevent their loss by diffusion to the outside environment. Negatively charged phosphorylated compounds cannot diffuse through the phospholipid bilayer. Thus, the catabolism of sugars always comprises a phosphorylation step. For example, sugars such as D-glucose, D-fructose, D-threalose, L-rhamnulose, D-xylose and L-arabinose are trapped in the cytoplasm as D-glucose-6-phosphate, D-fructose-1-phosphate, D-threalose-6-phosphate, L-rhamnulose-6-phosphate, D-sorbitol 6-phosphate, D-xylulose-5-phosphate and D-ribulose-5-phosphate, respectively. The same applies to the other carbon sources assimilable by microorganisms, such as polyols: mannitol, sorbitol and glycerol, respectively phosphorylated to mannitol-1-phosphate, sorbitol-6-phosphate and glycerol-3-phosphate. Externalizing the phosphorylation reaction makes it possible to solve this export problem and to carry out a microbial production of these compounds, in accordance with the present invention.

To do this, a strain of Escherichia coli was transformed to express in its periplasm a kinase catalyzing the phosphorylation of a sugar (first module). This reaction consumes a cofactor, ATP, and forms ADP as a coproduct. A second module was constructed by transforming a strain of Escherichia coli to express acetate kinase (AckA) in its periplasm. This enzyme catalyzes a reaction using acetyl phosphate and ADP to form acetate and ATP.

The reaction scheme is as follows:

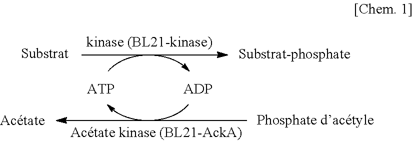

### [Text in Figure]

Acetate
Acetyl phosphate
Acetate kinase

The examples below illustrate the production of phosphorylated compounds of interest from three organic substrates (glycerol, glucose and fructose), with the intervention of cofactors ATP and ADP, corresponding to this reaction scheme. Due to the fact that the BL21-AckA module carrying out the regeneration of ATP is involved, the quantity of ATP introduced into the initial reaction mixture is markedly less than that of the substrate (in this case 32 times less for one of the examples and 60 times less for the other two).

The use of the BL21-AckA module thus makes it possible to achieve a substantial saving since the cost of ATP is around €300/kg. However, another important reason motivates the regeneration of the ATP cofactor. In fact, some kinases are inhibited by ADP. Glycerol kinase, for example, is inhibited by small quantities of ADP (Kic=500 μM). However, in the first reaction, one ADP molecule is generated per molecule of product formed. In case of inhibition by ADP, the production of the compound of interest is therefore impossible unless the ADP formed is continuously removed, which is done here by means of the reaction of regenerating ATP. The use of a regeneration module allows the production of phosphorylated molecules.

### Example 1.1: Production of Glycerol-3-Phosphate from Glycerol

Glycerol-3-phosphate is the entry point for the phospholipid pathway. This molecule of interest was produced in an amount of 325 mg/l microbially in a fed-batch bioreactor but its export proved to be a major problem (Popp et al. 2008). In addition, a phenomenon of glycerol dephosphorylation occurs inside the cell.

Two modules are used: the first module expresses glycerol kinase from E. coli which produces glycerol-3-phosphate from glycerol and ATP, the second module has the function of regenerating ATP via acetate kinase from E. coli AckA. Both the BL21-Glpk and BL21-AckA modules are obtained from a BL21(DE3) chassis.

Both modules are cultured and induced with IPTG in TB medium as described (Material and methods, point 3) before being recovered to carry out the production of glycerol-3-phosphate in a simple medium. The reaction mixture comprises:
Module 1: 2 ODU/ml BL21-Glk cells
Module 2: 2 ODU/ml BL21-AckA cells
Substrate: glycerol (160 mM)
Cofactor: ATP (5 mM)
Complementary substrate: acetyl phosphate (220 mM)
Buffered solution: HEPES (50 mM) and MgSO₄ (15 mM)

The glycerol-3-phosphate production reactions are carried out in volumes of 2 ml of reaction mixture by resuspension of the pellet containing an equivalent quantity of Glpk and AckA cells of 2 ODU in a volume of 2 ml. The tubes are then placed at 25° C. with stirring. Samples are taken in the reaction mixture after the addition of enzyme at 0 hours, and after 1 hour, 3 hours, 5 hours and 22 hours. The results are presented in FIG. 1. After 22 h of reaction, a production of 1.4 g/l glycerol-3-phosphate was obtained.

### Influence of the Cell Concentration on the Yield

The same protocol was repeated with a doubled module concentration. A production of 12 g/l was obtained in 23 h, corresponding to a yield of 81.5%, or 37 times more than the maximum production obtained by microbial route (Popp et al. 2008). Thus, the concentration of cells has an important influence and can be optimized.

### Influence of the Cell Concentration on the Reaction Rate

In order to evaluate the effect of the quantity of cells on the rate of production, different concentrations were used, under the same experimental conditions. The glycerol-3-phosphate concentrations obtained in one hour show that the rate of the reaction is proportional to the concentration of cells (FIG. 2a). Productivities ranging from 1 g/l/h to 4 g/l/h can therefore be obtained. These yields indicate that a scaling up of the process according to the invention is possible.

This example demonstrates the possibility of producing phosphorylated polyols by the process that is the object of the invention. This helped overcome the obstacles related to export and dephosphorylation by intracellular phosphatases.

### Influence of Reaction Volume

The processes according to the invention are intended to produce molecules at different scales, from the laboratory to the industrial level. To evaluate the effect of increasing the volume of the reaction mixture on glycerol-3-phosphate production, the reaction was carried out in volumes of 2 ml, 20 ml, 200 ml and 2000 ml. The modules used are, as previously, BL21-Glk and BL21-AckA. The reaction mixture is composed of:
Module 1: 2 ODU/ml BL21-Glk cells
Module 2: 2 ODU/ml BL21-AckA cells
Substrate: glycerol (25 mM)
Cofactor: ATP (5 mM)
Complementary substrate: acetyl phosphate (25 mM)
Buffered solution: HEPES (50 mM) and MgSO₄ (15 mM)

Samples were taken after adding cellular biocatalysts (modules) to the mixture at 0 h, then at 1 h, 3 h, 4 h and 5 h of reaction. Under the conditions described, with 25 mM acetyl phosphate, the theoretical maximum glycerol phosphate production is 4.3 g/l. The production kinetic profiles observed at 2 ml and 20 ml are similar. The maximum production is 3.3 g/l after 3 h with a yield of 76%. When the reaction volume is 200 ml or 2000 ml, 100% of the yield is reached after 3 h. These results highlight the transposable character of the process, an essential prerequisite for scaling up.

And at 2000 ml the product is stable. According to these results, scaling up the process by a factor of 1000 is quite possible, and it is even favorable to production.

### Example 1.2: Production of Glycerol-6-Phosphate from Glucose

Glucose-6-phosphate is an entry point for the glycolysis pathway and the pentose phosphate pathway. For this production, two protocols were tested, one with free enzymes in the periplasm, the other with the same enzymes anchored to the inner membrane.

Protocol with Free Enzymes

The two modules used are the BL21-Glk module expressing Escherichia coli Glk kinase, and the BL21-AckA ATP regeneration module where Escherichia coli acetate kinase is expressed. The enzymes are found in the free state in the periplasm. The two modules are cultured and induced with IPTG in TB medium, then introduced into the reaction mixture medium in an amount of 2 ODU in 2 ml. A control sample comprising strain BL21-WT is prepared. The reaction mixture comprises:
Module 1: 2 ODU/ml BL21-Glk cells
Module 2: 2 ODU/ml BL21-AckA cells
Substrate: 300 mM glucose
Cofactor: 5 mM ATP
Complementary substrate: 125 mM acetyl phosphate
Buffered solution: HEPES (50 mM) and MgSO₄ (15 mM)

FIG. 3a shows that glucose-6-phosphate is indeed synthesized, with a concentration of 30.8 g/L obtained from 5 h of reaction, i.e. a yield of 96%, limited by the quantity of acetyl phosphate (125 mM). A plateau is thus reached quickly. After 22 h, a decrease in glucose-6-phosphate is observed, due to degradation of the molecule. No production is observed with the control strain.

Protocol with Anchored Enzymes

The modules used here (BL21-nlpA-Glk and BL21-nlpA-AckA) express the same two enzymes (glucokinase Glk and acetate kinase AckA), but these are fused to the anchor peptide nlpA, so that both enzymes are anchored to the inner membrane of the periplasm in their respective modules. The modules are cultured and tested according to the same protocol as above.

The reaction scheme is as follows:

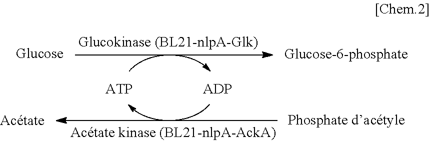

### [Text in Figure]

Acetate
Acetyl phosphate
Acetate kinase

FIG. 3b shows a production of glucose-6-phosphate at a concentration of 24.8 g/l obtained after 3 hours of reaction, i.e. a yield of 76%, limited by the quantity of acetyl phosphate (125 mM). After 22 h, a decrease in glucose-6-phosphate is observed, due to degradation of the molecule. No production is observed with the control strain.

Conclusion: Production reactions are possible when the enzymes are free or anchored. In this example the production of glucose-6-phosphate from anchored enzymes is faster than from free enzymes but the yield is lower. The process according to the invention can implemented with transformed bacteria generating periplasmic enzymes, either anchored or free.

### Example 1.3: Production of Glycerol-1-Phosphate from Fructose

Fructose-1-phosphate is a sugar phosphorylated in position 1 from which glyceraldehyde, a synthon of interest, can be generated. Its biological synthesis has never been described.

As previously, two modules are necessary. One carries the sequence encoding Homo sapiens KhkC fructokinase which is capable of phosphorylating sugars at position 1 (BL21-KhkC). The other expresses Escherichia coli acetate kinase responsible for regenerating the ATP cofactor (BL21-AckA already described).

For this test, 4 ODU/ml of each module were added to the reaction mixture, i.e., a total of 8 ODU in 2 ml. A control sample comprising strain BL21-WT is prepared. The reaction mixture comprises:
Module 1: 2 ODU/ml BL21-KhkC cells
Module 2: 2 ODU/ml BL21-AckA cells
Substrate: 300 mM fructose
Cofactor: 5 mM ATP
Complementary substrate: 125 mM acetyl phosphate
Buffered solution: HEPES (50 mM) and MgSO₄ (5 mM)

A control sample comprising strain BL21-WT (2 ODU/ml otherwise called OD_{600 nm}=2) is prepared.

The results presented in FIG. 4 show that the reaction is almost complete after 5 hours. It reaches a plateau after 22 hours with a concentration of 1.3 g/l of fructose-1-phosphate. The reaction is limited by acetyl phosphate (125 mM). No production is obtained with the control strain.

Conclusion: The process according to the invention is thus suitable for the production of phosphorylated sugars. Firstly, it is established that the production of phosphorylated sugars by externalizing phosphorylation reactions in the periplasm is possible and efficient. Secondly, the use of the same strain (BL21-AckA) in several reaction schemes illustrates the flexibility of the process and its economic interest, by limiting the biomolecular engineering work to obtain the required modules. Finally, the advantage of a module designed for the regeneration of the cofactor is that it can use low concentrations of ATP for the production of phosphorylated molecules. It is therefore possible to design reaction schemes using ATP naturally present in small quantities in the periplasmic compartment.

### Example 2: Conservation and Reuse of Cells

In the process according to the invention, the use of modules implemented in a manner decoupled from the prior cell culture phase makes it possible to carry out several reaction cycles with the same biomass and to preserve this biomass over time.

Glucose-6-phosphate production was carried out by reusing the cells of the BL21-GLK and BL21-AckA modules which had already been used in a previous production. To this end, a first production was carried out in which the cells were suspended in 2 ml of reaction mixture (8 ODU for each cell type). Then the cells used for this first production were separated from the supernatant, and the modules were separated by centrifugation. The pellets were recovered and stored at 4° C. After four days, the cells were resuspended in water and introduced into a new volume of the reaction mixture of 2 ml in an amount of 8 ODU for each cell type.

The reaction mixture used for both cycles consists of:
Module 1: 8 ODU/ml BL21-GlK cells
Module 2: 8 ODU/ml BL21-AckA cells
Substrate: 300 mM glucose
Cofactor: 5 mM ATP
Complementary substrate: 220 mM acetyl phosphate
Buffered solution: HEPES (50 mM) and MgSO₄ (5 mM)

The results presented in FIG. 5 show a production of 50 g/l of glucose-6-phosphate in 6 hours. The reaction would be complete if the final concentration obtained was 65 g/l. The reaction yield is therefore 75%. By reusing the cells, 1.6 times more glucose-6-phosphate was produced than when the cells were first used where 30 g/L was produced. The process according to the invention therefore makes it possible to preserve and use the same biocatalyst several times without loss of efficiency, and thus to reduce the cost associated with biomass production.

### Example 3: Production of Dihydroxyacetone from Glycerol

Dihydroxyacetone (DHA) is a tanning agent widely used in the cosmetic industry. The process according to the invention was used to produce DHA. It includes the recycling of redox cofactors involved in the reaction.

To do this, two modules were built. A strain of Escherichia coli was transformed to express in its periplasm an Escherichia coli glycerol dehydrogenase catalyzing the conversion of glycerol to DHA (first BL21-GldA module). This reaction consumes a cofactor, NAD+ and forms NADH as a coproduct. A second module (BL21-Nox module) was constructed by transforming a strain of Escherichia coli to express Lactobacillus sanfranciscensis NADH reductase (NOX) in its periplasm. This enzyme catalyzes a reaction using oxygen (O₂) and NADH to form water (H₂O) and NAD+, which is regenerated to intervene in the first reaction. Plasmid pET26b carrying the nox gene of L. sanfranciscensis which codes for NADH oxidase NOX was ordered from GenScript and transformed into strain BL21-WT.

The reaction scheme is as follows:

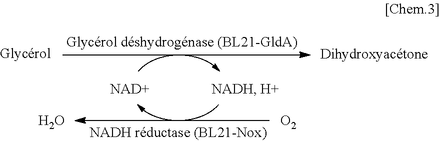

### [Text in Figure]

Glycerol dehydrogenase
Glycerol
Dihydroxyacetone
NADH reductase

Both modules are cultured and induced with IPTG in TB medium before being recovered to carry out the production of DHA. The reaction mixture comprises:
Module 1: 4 ODU/ml BL21-GldA cells
Module 2: 4 ODU/ml BL21-Nox cells
Substrate: 20 mM Glycerol
Cofactor: 5 mM NAD+
Buffered solution: 100 mM (Tris pH 8 or Gly pH 9).

Negative controls without substrate were prepared. In addition, negative controls where each strain is replaced by BL21(DE3) cells not expressing GldA or NOX enzymes were prepared. Since the optimum pH of GldA is 9 while that of NOX is 7.5, tests were carried out at pH 8 and 9 in order to find the best compromise for the functioning of the reaction. The measurements were carried out after 5 hours and are shown in FIG. 6.

A production of DHA is obtained in the presence of the two modules, at pH 8 and at pH 9, whereas no production appears in the wild-type control. A quantity of DHA of 260 mg/l at pH 8 and 330 mg/l at pH 9 was obtained, demonstrating the feasibility of the proposed reaction scheme.

### Example 4: Production of Glycolaldehyde from Glyoxal

Glycolaldehyde is a highly reactive molecule present in cellular metabolism and can be used like most aldehydes as a platform molecule. Nevertheless, it is too expensive for industrial use. Because of their reactivity, which makes them toxic to cells and unstable, aldehydes are rarely produced microbially. Their export is further complicated by their ability to react with membrane proteins and lipids. Glycolaldehyde could be obtained by reducing glyoxal, which is inexpensive but clearly toxic.

To do this, two modules were built. A strain of Escherichia coli was transformed to express Bacillus subtilis glyoxal reductase YvgN in its periplasm, catalyzing the conversion of glyoxal to glycolaldehyde (first BL21-YvgN module). This reaction consumes a cofactor, NADPH, and forms NADP as a coproduct (Dudek et al., 2013). A second module (BL21-PtxD) was constructed by transforming a strain of Escherichia coli to express in its periplasm phosphite dehydrogenase PtxD which is an NADP reductase. The ptxD gene of Pseudomonas stutzeri which codes for this enzyme comprises 18 mutations ensuring increased catalytic efficiency, stability at 37° C. and the management of the two substrates NAD and NADP. The Bacillus subtilis yvgn gene codes for glyoxal reductase (GR). The wild-type version was used. Expression plasmids pET22b-PtxD mutant and pET26b-YvgN were ordered from Genescript. The ptxD gene was ordered codon optimized for expression in E. coli. Plasmids pET22b and pET26b possess the signal sequence pelB responsible for the export of proteins into the periplasm; pET22b is resistant to ampicillin and pET26b to kanamycin. These plasmids were then transformed in BL21-WT to give the BL21-YvgN and BL21-PtxD modules.

The reaction scheme is as follows:

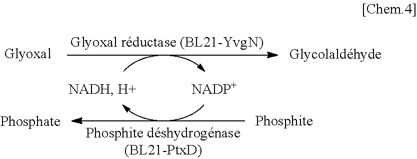

### [Text in Figure]

Glyoxal reductase
Glycolaldehyde
Phosphite dehydrogenase

The reaction mixture comprises:
Module 1: 1 ODU/ml BL21-YvgN cells.
Module 2: 1 ODU/ml BL21-PtxD cells (except for pre-testing)
   Substrate: 10 mM glyoxal

### Cofactor: 0.4 mM NADPH

Complementary substrate: 10 mM phosphite
Buffered solution: 50 mM HEPES pH 7.5 and 10 mM KCl.

In a preliminary test, the production of aldehyde by periplasmic glyoxal reductase was carried out alone, without a regeneration system. In a second step, the reactions are initiated by the addition to the reaction mixture of BL21-PtxD induced cells at a concentration of 1 ODU, and phosphite at 10 mM in order to regenerate the NADP produced into NADPH. The reactions are placed at a temperature of 37° C. with stirring at 200 rpm. Measurements are made at 0 hours, 1 hour, 3 hours and 5 hours.

The results presented in FIG. 7 show that glycolaldehyde is detected as early as 1 h of reaction with and without a regeneration system, but the production already reaches a plateau in the absence of PtxD due to the depletion of NADPH. On the contrary, in the presence of the BL21-PtxD module, the reaction continues thanks to the regeneration of the NADPH. After 5 h, glycolaldehyde production is 25 times higher, for a yield of 37%.

The BL21-YvgN and BL21-PtxD modules therefore efficiently produce glycolaldehyde from glyoxal in the periplasmic compartment, with no adverse effect on bacterial cells. This demonstrates that it is possible by the process according to the invention to produce aldehyde compounds from a toxic precursor.

### Example 5: Production of an Alcohol from an Amino Acid

A reaction scheme has been developed to produce 2-phenylethanol from L-phenylalanine. This reaction scheme is based on the use of four separate modules. Three modules were constructed which, respectively, express the S. cerevisiae ARO8, ARO10 and ADH5 genes, the first coding for L-phenylalanine oxoglutarate aminotransferase, the second for phenylpyruvate carboxylase, and the third for 2-phenylethanol dehydrogenase. A fourth module expressing phosphite dehydrogenase PtxD is designed for NADH regeneration.

The reaction scheme is as follows:

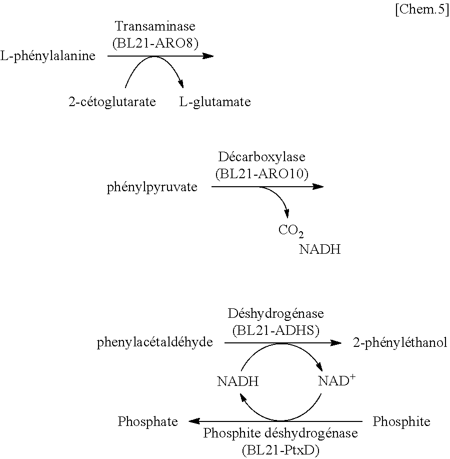

### [Text in Figure]

Decarboxylase
Dehydrogenase
Phenylalanine phenylpyruvate phenylacetaldehyde 2-phenylethanol
Phosphite dehydrogenase

It is here the third reaction producing the compound of interest which gives rise to coupling with regeneration of the cofactor NADH from the coproduct NAD+.

The reaction mixture is as follows:
Module 1: 5 OD/ml BL21-ARO8 cells
Module 2: 5 OD_{600 nm}/ml BL21-ARO10 cells
Module 3: 5 OD_{600 nm}/ml BL21-ADH5 cells
Module 4: 5 OD_{600 nm}/ml BL21-PtxD cells
Substrate: L-phenylalanine (10 mM)
Cofactors: 2-ketoglutarate (10 mM), MgCl₂ (5 mM), ZnCl₂ (5 mM), thiamine (0.2 mM), pyridoxal (0.1 mM), NADH (1 mM)
Complementary substrates: phosphite (100 mM)
Buffered solution: HEPES pH 7 (100 mM).

The 2 ml reactions were placed at 33° C. and stirred at 200 rpm for 5 h. Samples were taken at 0, 1, 3 and 5 h.

The synthetic consortia of the four bacterial biocatalysts BL21-ARO8, BL21-ARO10, BL21-ADH5 and BL21-PtxD was used to produce 2-phenylethanol from L-phenylalanine. The efficacy of this consortia was tested and validated by measuring the appearance of 2-phenylethanol by HPLC. [FIG. 13] The results showed that the synthesis of 65 mg/l of 2-phenylethanol was obtained in 5 hours of reaction.

### Example 6: Production of Rare Sugar Isomers

Rare sugars are produced mainly by isomerization of a common sugar. The difficulty originates from the fact that isomerization is a reversible reaction. The objective here is to limit the reverse reaction by transforming the rare sugar formed into a new compound, for example phosphorylated sugar, which is then dephosphorylated to obtain the isomer of interest.

In this example, a first reaction isomerizes a simple sugar, D-xylose, into a rare sugar, D-xylulose. A second reaction phosphorylates D-xylulose to D-xylulose-5-phosphate in the presence of ATP acting as a cofactor. The reaction scheme continues with two downstream reactions, i.e., a reaction consuming the ADP formed and acetyl phosphate to regenerate ATP, and a dephosphorylation reaction of D-xylulose-5-phosphate, giving back D-xylulose.

Four different modules are used.
The first module (BL21-XylA) is a strain of Escherichia coli transformed to express in its periplasm the xylose isomerase XylA present in E. coli.
The second module (BL21-XylB) is a strain of Escherichia coli transformed to express a kinase, XylB of E. coli, which phosphorylates xylulose to xylulose-5P by consuming an ATP molecule.
The BL21-AckA module already seen above is used to regenerate ATP.
The fourth module (BL21-AphA) is a strain of Escherichia coli transformed to express E. coli broad spectrum phosphatase AphA in its periplasm (Passariello et al., 2006).

The complete reaction scheme is presented below.

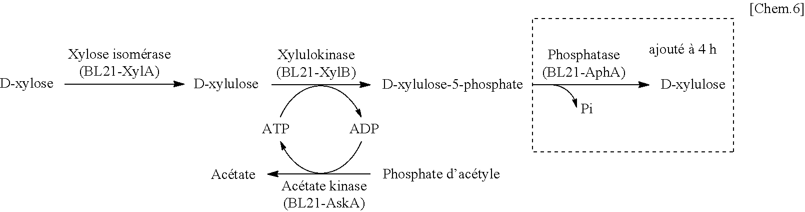

### [Text in Figure]

Xylose isomerase
Added at 4 h
Acetyl phosphate
Acetate kinase

Both modules are cultured and induced with IPTG in TB medium before being recovered to carry out the production of D-xylose. The reaction mixture comprises:
Module 1: 8 ODU/ml BL21-XylA cells
Module 2: 8 ODU/ml BL21-XylB cells
Module 3: 8 ODU/ml BL21-AckA cells
Module 4: 8 ODU/ml BL21-AphA cells
Substrate: D-xylose (300 mM)
Cofactor: ATP (5 mM)
Complementary substrate: acetyl phosphate (125 mM)
Buffered solution: HEPES pH 7.5 (50 mM), MgSO₄ (5 mM), MnSO₄ (3 mM)

The process is initiated with modules 1, 2 and 3 each expressing an enzyme in the reaction mixture. The tubes are placed at a temperature of 37° C. with stirring at 200 rpm. Module 4 expressing AphA phosphatase is added 4 hours after the beginning of the reaction and the pH is adjusted to 6. The reaction is monitored at 7 hours and then at 22 hours. In parallel, a reaction is carried out with the BL21-Xyla module alone, in order to evaluate the simple isomerization reaction. The evolution of the concentrations of D-xylose (FIG. 8) and of D-xylulose (FIG. 9) during the reaction was monitored by HPIC.

FIG. 8 shows that the D-xylose substrate is consumed in similar amounts in both protocols. However, after 4 hours, the production of D-xylulose was two times lower for the complete reaction than for the isomerization reaction alone (FIG. 9). This difference is explained by the consumption of D-xylulose due to the production of D-xylulose-5P in the complete scheme. Its presence is not detectable by the D-xylulose assay method but has been confirmed by mass spectrometry.

Module 4 carrying the phosphatase AphA, added to the reaction mixture at T=4 h, catalyzes the dephosphorylation of D-xylulose-5P to D-xylulose. Thus, 3 hours and 18 hours after the introduction of the phosphatase, an increase of 2 g/m and 8 g/l of D-xylulose, respectively, is observed. The amount of D-xylulose in the complete reaction (12.8 g/l) exceeds that in the isomerization reaction alone (10.2 g/l), resulting in a significant gain of 2.6 g/l.

This is explained by the fact that during the isomerization reaction an equilibrium is created between the two species D-xylose and D-xylulose, so that there can be no total conversion of D-xylose. The presence of the BL21-XylB kinase module in the reaction mixture makes it possible to trap D-xylulose in phosphorylated form, which can then no longer be converted to D-xylose. D-xylulose-5P accumulates in the reaction mixture, and the addition of BL21-AphA phosphatase allows for increased recovery of D-xylulose compared to simple isomerization. By pulling isomerization to a phosphorylated form of the rare sugar, a higher conversion rate is obtained.

These results first show that isomerization, phosphorylation and dephosphorylation by the BL21-XylA, BL21-XylB and BL21-AphA modules are possible. They also attest that four different modules worked together to achieve a complex reaction scheme, including a mechanism for ATP regeneration. The synergistic and sequential use of all kinds of modules in the context of the process that is the object of the invention is thus validated.

### Example 7: Production of D-Fructose-1,6-Bisphosphate from D-Xylose

The conversion of D-xylose to D-fructose-1,6-bisphosphate is part of the pentose phosphate pathway, which is the natural assimilation pathway of D-xylose into the cytoplasm. This conversion was carried out by a reaction scheme implementing nine reactions carried out by eight different modules.

The eight modules used are constructed from a strain of Escherichia coli, each transformed to express in its periplasm an enzyme present in E. coli:
1—xylose isomerase XylA, which isomerizes D-xylose to D-xylulose
2—xylulose kinase XylB, which phosphorylates D-xylulose to D-xylulose-5P
3—acetate kinase AckA, which regenerates ATP from ATP and acetyl phosphate
4—ribulose-phosphate-3-epimerase Rpe, which converts D-xylulose-5P to D-ribulose-5P
5—ribose-5-phosphate isomerase RpiA, which produces D-ribose-5P from D-ribulose-5P
6—transketolase Tkt, which converts D-xylulose-5P and D-ribose-5P to D-glyceraldehyde-3P and D-sedoheptulose-7P
7—transaldolase Tal, which converts D-glyceraldehyde-3P and D-sedoheptulose-7P to D-fructose-5P and D-erythrose-4P
8—6-phosphofructokinase 1 PfkA which converts D-fructose-5P to D-fructose-1,6bP The complete reaction scheme is presented below.

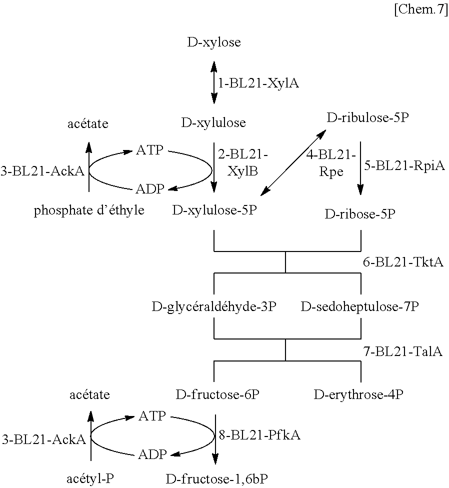

### [Text in Figure]

Acetate
Ethyl phosphate
D-glyceraldehyde-3P
Acetyl-P

The presence of enzymes in the periplasm of the eight modules was verified by recovering the periplasmic fraction by osmotic shock for each of the strains. The size of the enzymes and the quantity of cells used for the osmotic shock (OD 600 nm multiplied by the culture volume) are given in Table 4.

TABLE 4
Molecular weight in kilodalton of the proteins XylA,
XylB, AckA, Rpe, RpiA, TktA, TalA and PfkA and optical
density of the culture used for osmotic shock

| Protein | XylA | XylB | Ack | Rpe | RpiA | TktA | TalA | PfkA |
|---|---|---|---|---|---|---|---|---|
| Size | 44-49 | 52 | 43.3 | 24 | 22-27 | 73 | 35 | 35 |
| (kDa) | | | | | | | | |
| OD 600 | 2.84 | 2.04 | 9.2 | 2.38 | 1.86 | 4.46 | 1.34 | 8.74 |
| nm | | | | | | | | |

The results of the analysis by SPS-PAGE gel are given in FIG. 10. They show that the expression in the periplasm of the enzymes expected for the 8 modules is validated.

The modules were cultured and induced with IPTG in TB medium before being recovered to produce D-fructose-1,6bP in the reaction mixture comprising:
Module 1: 8 ODU/ml BL21-XylA cells
Module 2: 8 ODU/ml BL21 XylB cells
Module 3: 8 ODU/ml BL21 AckA cells
Module 4: 8 ODU/ml BL21-Rpe cells
Module 5: 8 ODU/ml BL21-RpiA cells
Module 6: 8 ODU/ml BL21-Tkt cells
Module 7: 8 ODU/ml BL21-Tal cells
Module 8: 8 ODU/ml BL21-PfkA cells
Substrate: 100 mM of D-xylose
Cofactor: 3 mM ATP
Complementary substrate: 100 mM acetyl phosphate
Buffered solution: HEPES pH 7.5 (50 mM), MgSO₄ (20 mM), MnSO₄ (1 mM) and thiamine pyrophosphate (1 mM) COCl₂ (0.1 mM)

The determination of D-fructose-1,6bP was carried out immediately after addition of the modules to the reaction mixture, after 1 hour, 3 hours and 4 hours. Two controls were carried out, one without substrate and the other without module (the eight modules are replaced by BL21 WT cells. The results are presented in FIG. 11. After 1 h of reaction, the production of D-fructose-1,6-bisphosphate is 1.3 g/l and reaches a maximum of 2.8 g/l in 3 h. No production of D-fructose-1,6-bisphosphate is measured in the two control reactions.

D-sedoheptulose-7-phosphate was also assayed (FIG. 12). Indeed, the XylB and PfkA carrier modules catalyze the only irreversible reactions in the reaction scheme, and D-sedoheptulose-7-phosphate is in the middle of the reaction chain composed of reversible reactions whose products equilibrate with each other. D-sedoheptulose-7-phosphate was assayed immediately after adding the modules to the reaction mixture and after 1 hour, 3 hours and 4 hours. It is at 130 mg/l after 4 hours in the complete reaction and is absent from the control samples. The other intermediate compounds are difficult to detect with the analytical methods used, but the level of D-sedoheptulose-7P is representative of their concentration, which is therefore similar. The production of D-fructose-1,6-bisphosphate according to a complex reaction scheme was thus successfully carried out.

These results demonstrate that the process according to the invention based on the externalization of reactions works efficiently, even for a long and branched chain, involving many modules. It is also particularly advantageous to have been able to reconstitute a cytoplasmic naturally complex metabolic pathway in this way.

### Example 8: Effect of a Consortium of 2 Bacteria Each Expressing 1 Enzyme, Compared to One Bacterium Expressing 2 Enzymes, on the Production of a Product (G3P)

Glycerol-3-phosphate production is based on the catalytic activity of glycerol kinase and acetate kinase. In order to demonstrate the value of using a synthetic consortia (2 bacteria each expressing a different enzyme), compared to a bacterium expressing both enzymes, a whole-cell biocatalyst was created co-expressing Escherichia coli glycerol kinase GlpK and Escherichia coli acetate kinase AckA in its periplasm. To do this, a plasmid pETDuet-pelB-glpK-pelB-ackA containing two cloning sites under the control of two T7 promoters was constructed and transformed in E. coli. This biocatalyst is referred to herein as BL21-GLK-AckA. The design of the vector pETDuet was carried out so that the expression cassettes of pETDuet were identical to those of pET22b. Thus, the comparison of strains BL21-AckA, BL21-GlpK and BL21-GlpK-AckA has as its object the effect of co-expression without influence of the promoter, RBS or signal peptide.

The periplasmic fractions of strains BL21-AckA and BL21-AckA-GlpK were recovered after osmotic shock from 2 ml of cell suspension concentrated at 2 OD_{600 nm} for each of the strains. We observe that the 43 kDa AckA band is at least two times lower in BL21-AckA-GlpK compared to BL21-AckA. This result demonstrates a negative effect of the co-expression of two enzymes in the same bacterium on the production of the enzyme AckA in the periplasm of the biocatalyst BL21-AckA-GlpK (1 bacterium expressing 2 enzymes). By comparing the 56 kDa GlpK bands of strains BL21-GlpK and BL21-GlpK-AckA, we also observe a negative effect of co-expression of the 2 enzymes in the same bacterium on periplasmic GlpK production.

Finally, we extracted the periplasmic fractions from a mixture of BL21-AckA and BL21-GlpK (consortium of bacteria according to the invention), where each of the strains was at a concentration of 1 OD_{600 nm}, condition approaching standard co-culture conditions. The AckA and GlpK bands are more marked in the co-culture (consortium according to the invention), compared to the periplasmic fraction extracted from the biocatalyst co-expressing AckA and GlpK (1 bacterium expressing 2 enzymes). [FIG. 14] These results show that the use of a consortium of bacteria makes it possible to obtain a better expression of each enzyme, compared to the use of the same bacterium co-expressing both enzymes.

We also observe that the signal peptide pelB allows a better production of the enzyme AckA than of the enzyme GlpK in the periplasm of strains BL21-AckA and BL21-GlpK, respectively. This observation is not surprising since the effectiveness of signal peptides depends on the target protein. It is possible to modulate the amount of BL21-GlpK and BL21-AckA in the glycerol-3P production reaction mixture to maximize the efficiency of the reaction by increasing the amount of BL21-GlpK to compensate for the low efficiency of PelB. This strategy makes it possible to easily improve the production of glycerol-3P which is advantageous for industrial use. In comparison, the biocatalyst BL21-GlpK-AckA co-expressing the enzymes GlpK and AckA does not offer this modularity. Each of the expression cassettes (promoter and/or RBS and/or signal peptide) would have to be reconstructed to modulate the expression of GlpK and AckA; this is more complex and time-consuming.

We also compared the production of G3P by the biocatalyst BL21-GlpK-AckA (1 bacterium expressing 2 enzymes) and the synthetic consortia BL21-GlpK and BL21-AckA (according to the invention). The reaction mixture is composed of:

$$Module ( s ) : BL 21 - GlpK and / or BL 21 - AckA and / or BL 21 - GlpK - AckA cells ( {OD}_{600 nm total} = 2 )$$

Substrate: 160 mM glycerol
Cofactors: 5 mM ATP, 15 mM MgSO₄
Complementary substrate: 220 mM of acetyl phosphate
Buffered solution: 50 mM HEPES buffer at pH 7.5

The 5 ml reactions are placed at a temperature of 37° C. with stirring at 200 rpm and followed for 24 hours. Samples of 200 μL are taken at T0 h, T0.5 h, T1 h, T2 h, T3 h, T5 h and T24 h.

The BL21-GlpK-AckA biocatalyst is capable of producing 2.1 grams of G3P per liter in 24 hours. However, by using a mixture of two types of biocatalysts—BL21-GlpK and BL21-AckA in equal proportions (50% of each), it is possible to produce 5 grams of G3P per liter in 24 hours, or a production more than 2 times greater than in the system of 1 bacterium co-expressing 2 enzymes. And by adjusting the proportions of the two types of biocatalysts, it is possible to further optimize the production of G3P. Indeed, using a mixture of 92.5% BL21-GlpK and 7.5% BL21-AckA, the production of G3P reaches 10 grams per liter in just 5 hours. [FIG. 15]. These results show that by modulating the proportion of BL21-GlpK and BL21-AckA biocatalysts, it is possible to significantly improve G3P bioproduction. It is a considerable advantage of the use of consortia according to the invention to be able to easily modulate the proportions of each biocatalyst, in a much easier and more reliable way than through the use of complex genetic engineering within the same bacterium co-expressing several enzymes.

### Example 9: Production from Extracellular ATP

The purine adenosine 5-triphosphate (ATP) is not only a universal intracellular energy transporter, but also plays an important role as an extracellular signaling molecule. ATP is well known as a messenger for intercellular communications in multicellular organisms, but older phylogenetically unicellular organisms such as yeast or bacteria also use ATP as an extracellular signaling molecule. However, the mechanisms of ATP secretion by bacteria and its extracellular implications still need to be elucidated (Spari D and Beldi G, 2020).

Alvarez et al. showed that in E. coli DH5a ATP is rapidly hydrolyzed in the periplasmic space. The steady-state extracellular ATP concentration in the periplasmic space was determined to be 24±3 μm/10¹⁰ bacteria. (Alvarez et al., 2017).

In the context of the production of phosphorylated molecules, such as glucose-6-phosphate, ATP is an essential cofactor for phosphorylation. However, ATP is an expensive molecule to produce and often has to be regenerated from less expensive molecules, such as ADP.

The use of an ATP regeneration module such as BL21-AckA allows the trace amounts of ATP present in the periplasm to be reused for the production of phosphorylated molecules. This reduces production costs and makes efficient use of the natural resources of the cell.

G6P productions have thus been achieved according to the process of the invention, without the addition of commercial ATP.

The first reaction mixture comprises:
Module 1: 2 ODU/ml BL21-Glk cells
Module 2: 2 ODU/ml BL21-AckA cells
Substrate: 300 mM glucose
Complementary substrate: 250 mM acetyl phosphate
Buffered solution: HEPES (50 mM) and MgSO₄ (15 mM). When the cellular concentration of each biocatalyst is 2 OD 600 nm/ml, it is possible to produce 4 g/l of glucose-6-phosphate without the addition of ATP. This suggests that ATP production is sufficient to support glucose-6P production at this cell density.

The number of Escherichia coli cells per 1 OD 600 nm depends on many factors, including the growth conditions, the culture medium and the bacterial strain used. However, there is an empirical correlation between the optical density at 600 nm and the number of bacterial cells. In general, for E. coli, the optical density at 600 nm of 1 corresponds to a cell concentration of about 8×10⁸ cells/ml (https://www.agilent.com/store/biocalculators/calcODBacterial.jsp). Under the conditions of FIG. 16 where each biocatalyst is present at an optical density of 600 nm of 2/ml, there would be 3.2×10⁹ cells/ml. Considering the concentration of ATP 24±3 μm/10¹⁰ bacteria provided by Alvarez et al., the concentration of ATP in the test would be 7.68 μm. This quantity of ATP allowed the synthesis of 4 g/L glucose-6P or 15,000 μm; the BL21-AckA module would have made it possible to exceed 2000 times the possible production from naturally present extracellular ATP. These results show that the process of the invention makes it possible to optimize a trace cofactor for bioproduction, which is particularly interesting from an economic point of view.

### REFERENCES

Alvarez, C. L., G. Corradi, N. Lauri, I. Marginedas-Freixa, M. F. Leal Denis, N. Enrique, S. M. Mate, V. Milesi, M. A. Ostuni, V. Herlax, and P. J. Schwarzbaum. 2017. “Dynamic regulation of extracellular ATP in Escherichia coli.” Biochem J 474 (8):1395-1416. doi: 10.1042/bcj20160879.
Brass, J. M, C. F. Higgins, M. Folley, P. A. Rugman, J. Birmingham, and P. B. Garland. 1986. Lateral diffusion of proteins in the periplasm of Escherichia coli. J. Bacteriol. 165:787-794.
Chung, C. T., and R. H. Miller. 1993. “Preparation and storage of competent Escherichia coli cells.” Methods Enzymol 218:621-7. doi: 10.1016/0076-6879(93)18045-e.
Crans, Debbie C., and George M. Whitesides. 1983. “A convenient synthesis of disodium acetyl phosphate for use in situ ATP cofactor regeneration.” The Journal of Organic Chemistry 48 (18):3130-3132. doi: 10.1021/jo00166a048.
Dudek, Hanna M., Petra Popken, Edwin van Bloois, Wouter A. Duetz, and Marco W. Fraaije. 2013. “A Generic, Whole-Cell-Based Screening Method for Baeyer-Villiger Monooxygenases.” Journal of Biomolecular Screening 18 (6):678-687. doi: 10.1177/1087057113480390.
Eichmann J, Oberpaul M, Weidner T, Gerlach D, Czermak P. Selection of High Producers From Combinatorial Libraries for the Production of Recombinant Proteins in Escherichia coli and Vibrio natriegens. Front Bioeng Biotechnol. 2019 Oct. 4; 7:254. doi: 10.3389/fbioe.2019.00254. PMID: 31637238; PMCID: PMC6788121.
Karyolaimos A, Ampah-Korsah H, Hillenaar T, Mestre Borras A, Dolata K M, Sievers S, Riedel K, Daniels R, de Gier J W. Enhancing recombinant protein yields in the E. coli periplasm by combining signal peptide and production rate screening. Front Microbiol. 2019; 10:1511.
Mergulhao F J, Monteiro G A. 2007. Periplasmic targeting of recombinant proteins in Escherichia coli. Methods in Molecular Biology 390:47-61.
Passariello, C., C. Forleo, V. Micheli, S. Schippa, R. Leone, S. Mangani, M. C. Thaller, and G. M. Rossolini. 2006. “Biochemical characterization of the class B acid phosphatase (AphA) of Escherichia coli MG1655.” Biochim Biophys Acta 1764 (1):13-9. doi: 10.1016/j.bbapap.2005.08.028.
Popp, A., H. T. Nguyen, K. Boulahya, C. Bideaux, S. Alfenore, S. E. Guillouet, and E. Nevoigt. 2008. “Fermentative production of L-glycerol 3-phosphate utilizing a Saccharomyces cerevisiae strain with an engineered glycerol biosynthetic pathway.” Biotechnol Bioeng 100 (3):497-505. doi: 10.1002/bit.21777.
Spari D, Beldi G. Extracellular ATP as an Inter-Kingdom Signaling Molecule: Release Mechanisms by Bacteria and Its Implication on the Host. Int J Mol Sci. 2020 Aug. 4; 21(15):5590. doi: 10.3390/ijms21155590. PMID: 32759857; PMCID: PMC7432876.
Stock, J. B., B. Rauch, and S. Roseman. 1977. Periplasmic space in Salmonella typhimurium and Escherichia coli. J. Biol. Chem. 252:7850-7861. [symbol]
\px%OH%X qt shij|xp%OQ3%uM %kymj%˜ytuqfxr %fsi%jwuqfxr %tk%Jxhmjwdhmrf% htq%wfuni %r jfxzwjr jsy%g˜%lwjjs% qztwjxhjsy%wtyrs %qztwr jyw˜3%O%Gfhyjwtq% 755<%F sl@6=>−6:; 562<3%itn%653667=4OG355; 6:25<%Juzg%755<%Ozs%63%UR NI ?% 6<: 9:7>7@uR HNI %JR H6>: 6=6>3
Zhou Y, Wang L, Yang F, Lin X, Zhang S, Zhao Z K. Determining the extremes of the cellular NAD(H) level by using an Escherichia coli NAD(+)-auxotrophic mutant. Appl Environ Microbiol. 2011 September; 77(17):6133-40. doi: 10.1128/AEM.00630-11. Epub 2011 Jul. 8. PMID: 21742902; PMCID: PMC3165392.

1. A process for producing a compound of interest from an organic substrate, comprising the steps of:
a) preparing a reaction mixture comprising, in a suitable medium,
n bacteria, n being an integer at least equal to 2, each bacterium being genetically modified to express in its periplasmic space at least one enzyme E1, E2, . . . En, each of said n bacteria expressing an enzyme different from the other bacteria,
an enzyme E1 being able to catalyze a first reaction R1 from said organic substrate to provide a first product P1,
each of said enzymes E2, . . . , En, being capable of catalyzing a reaction R2, . . . , Rn, from a product or a coproduct obtained by a reaction Rn−1, to provide, respectively, a product P2, . . . , Pn,
said organic substrate,
b) allowing the reaction mixture thus obtained to react, and
c) separating the biomass from the supernatant and extracting therefrom said compound of interest consisting of one of the products P1, P2, . . . , Pn.

2. The process according to claim 1, wherein said n genetically modified bacteria are Gram-negative diderm bacteria, each chosen from the Enterobacteriaceae, Alcaligenaceae, Vibrionaceae, or Pseudomonadaceae family.

3. The process according to claim 1, wherein each of the n bacteria is genetically modified to express at least one polypeptide comprising, respectively, one of said enzymes E1, E2, . . . , En, linked to a signal peptide addressing said polypeptide into the periplasmic space of said bacterium.

4. The process according to claim 3, wherein one or more of said n bacteria are genetically modified to express a polypeptide comprising, respectively, one of said enzymes E1, E2, . . . , En, linked to a membrane anchoring peptide including said signal peptide.

5. The process according to claim 1, wherein at least one of said n bacteria is genetically modified to express in its periplasmic space at least two enzymes En-a and En-b capable of catalyzing at least two reactions Rn-a and Rn-b, so that said reactions RN-a and RN-b occur in the periplasmic space of said at least one bacterium.

6. The process according to claim 1, wherein the reaction mixture comprises a bacterium expressing a first enzyme E1 capable of catalyzing the first reaction R1 from said organic substrate to provide a first product P1, and a bacterium expressing a second enzyme E2 capable of catalyzing a second reaction R2 from said first product P1, to form a second product P2, which is recovered in step c) as a compound of interest, or is consumed as a substrate of a third reaction.

7. The process according to claim 6, wherein the reaction mixture further comprises at least one bacterium expressing an enzyme E3, . . . , En, capable of catalyzing a reaction R3, . . . , Rn, from a product Pn−1 obtained by a reaction Rn−1, to provide a product Pn, which is recovered in step c) as a compound of interest or is consumed as a substrate of a reaction Rn+1.

8. The process according to claim 1, wherein the reaction mixture comprises a cofactor CoF1 of said organic substrate, a bacterium expressing an enzyme E1 capable of catalyzing a first reaction R1 from said organic substrate and the cofactor CoF1 to form a first product P1 and a first coproduct CoP1; and a bacterium expressing a second enzyme E2 capable of catalyzing a second reaction R2 from the first product P1 to form a second product P2, or from the first coproduct CoP1 to form a second coproduct CoP2, at least one of the two being consumed in a third reaction, or recovered in step c) as a compound of interest.

9. The process according to claim 8, wherein the reaction mixture comprises at least one cofactor CoFn, at least one bacterium expressing an enzyme capable of catalyzing a reaction Rn from the product Pn−1 of a reaction Rn−1 and said cofactor CoFn to form a product Pn and a coproduct CoPn, at least one of the two being consumed in a reaction Rn+1, or recovered in step c) as a compound of interest.

10. The process according to claim 9, wherein the reaction mixture can also comprise at least one substrate SCn complementary to said at least one cofactor CoFn.

11. The process according to claim 1, wherein said at least one cofactor CoFn can be introduced entirely or partially into the reaction mixture in step a), or during step b).

12. The process according to claim 1, wherein at least one cofactor CoFn is formed at least in part in the reaction mixture by a reaction Rn−1 catalyzed by an enzyme En−1 capable of forming a product Pn−1 and a coproduct CoPn−1, said coproduct CoPn−1 being identical to the cofactor CoFn.

13. The process according to claim 12, wherein the quantity of the first cofactor CoF1 introduced into the reaction mixture in step a) corresponds to a molar concentration at least 20 times lower than the initial molar concentration of said organic substrate.

14. The process according to claim 1, wherein the organic substrate of the first reaction is chosen from carbohydrates, aldehydes, alcohols, organic acids, carbamates, hydrocarbons, amino acids, and carbon dioxide.

15. The process according to claim 1, wherein the enzymes E1, E2, . . . , En are each chosen from kinases, dehydrogenases, phosphatases, reductases, isomerases, and transferases.

16. The process according to claim 1, wherein at least one of the enzymes E1, E2, . . . , En catalyzes a reaction providing one of the coproducts CoP1, CoP2, . . . , CoPn, chosen from ATP, ADP, AMP, UTP, UDP, UMP, NAD+, NADH, NADP+, NADPH, FAD, FADH₂, coenzyme A, or catalyzes a reaction using one of them as cofactor.

17. The process according to claim 1, wherein after step c) of the process that is the object of the present invention, the biomass separated from the supernatant is reused to prepare a new reaction mixture according to step a), with or without an intermediate preservation step.

18. The process according to claim 1, wherein steps a) and b) are carried out by adding to the reaction mixture containing said n bacteria, continuously or at intervals of time,
a cofactor of said organic substrate, a cofactor of said products obtained by a reaction Rn−1, called cofactors CoF1, CoF2, . . . , CoFn; a complementary substrate SC of a coproduct obtained by a reaction Rn−1, and by allowing the reaction mixture thus obtained to react continuously or semi-continuously.

19. A reaction mixture for the production of a compound of interest from an organic substrate, wherein it contains, in a suitable medium:
n bacteria, n being an integer at least equal to 2, each bacterium being genetically modified to express in its periplasmic space at least one enzyme E1, E2, . . . En, each of said n bacteria expressing an enzyme different from the other bacteria,
an enzyme E1 being able to catalyze a first reaction R1 from said organic substrate to provide a first product P1,
each of said enzymes E2, . . . , En, being capable of catalyzing a reaction R2, . . . , Rn, from a product or a coproduct obtained by a reaction Rn−1, to provide, respectively, a product P2, . . . , Pn, and optionally a coproduct CoP2, . . . , CoPn, and
said organic substrate.

20. A method for producing a compound of interest chosen from carbohydrates, aldehydes, alcohols, organic acids, carbamates, hydrocarbons, and amino acids, which comprises a step using the reaction mixture according to claim 19.
